(19) United States
(12) Patent Application Publication
(10) Pub. No.: US 2022/0054261 A1
(43) Pub. Date: Feb. 24, 2022

(54) MINIMAL FRAME PROSTHETIC CARDIAC VALVE DELIVERY DEVICES, SYSTEMS, AND METHODS

(71) Applicant: SHIFAMED HOLDINGS, LLC, Campbell, CA (US)

(72) Inventors: Claudio ARGENTO, Felton, CA (US); Andrew BACKUS, Campbell, CA (US); Ali SALAHIEH, Campbell, CA (US); Connor MULCAHY, Campbell, CA (US)

(73) Assignee: SHIFAMED HOLDINGS, LLC, Campbell, CA (US)

(21) Appl. No.: 17/599,710

(22) PCT Filed: Apr. 10, 2020
(86) PCT No.: PCT/US2020/027744

§ 371 Date: Sep. 29, 2021

Related U.S. Application Data
(60) Provisional application No. 62/833,425, filed on Apr. 12, 2019.
(60) Provisional application No. 62/925,505, filed on Oct. 24, 2019.

(51) Int. Cl. A61F 2/24; A61F 2/95

(52) U.S. Cl. CPC A61F 2/2418; A61F 2/95

(57) ABSTRACT

Disclosed herein are prosthetic valve devices, systems, and methods of installation of prosthetic valve devices and systems in a target region of a subject. Prosthetic valve devices disclosed herein comprise a frame structure having expanded and unexpanded configurations aiding in minimally-invasive delivery of the devices to the target region. The prosthetic valve devices include frame structures designed to minimize the amount of material used to form the devices without sacrificing structural strength. The prosthetic valve devices also include frame structures of minimal longitudinal length, allowing easier and more precise delivery and deployment of the devices.

### CROSS-REFERENCE

This application claims priority to U.S. Provisional Application No. 62/833,425, filed Apr. 12, 2019, entitled “Minimal Frame Prosthetic Cardiac Valve Delivery Devices, Systems, and Methods,” and U.S. Provisional Application No. 62/925,505, filed Oct. 24, 2019, entitled “Minimal Frame Prosthetic Cardiac Valve Delivery Devices, Systems, and Methods,” the entireties of which are incorporated by reference herein.

This application may also be related to U.S. patent application Ser. No. 16/546,901, filed Aug. 21, 2019, entitled “Prosthetic Cardiac Valve Devices, Systems, and Methods; U.S. patent application Ser. No. 16/594,946, filed Oct. 7, 2019, entitled “Prosthetic Cardiac Valve Devices, Systems, and Methods”; International Patent Application No. PCT/US2019/057082, filed Oct. 18, 2019, entitled “Adjustable Medical Device”; U.S. patent application Ser. No. 16/723,537, filed Dec. 20, 2019, entitled “Prosthetic Cardiac Valve Devices, Systems, and Methods” and International Patent Application No. PCT/US2020/023671, filed Mar. 19, 2020, entitled “Prosthetic Cardiac Valve Devices, Systems, and Methods,” the entireties of which are incorporated by reference in their entireties.

### BACKGROUND

Blood flow between heart chambers is regulated by native valves—the mitral valve, the aortic valve, the pulmonary valve, and the tricuspid valve. Each of these valves is a passive one-way valve that opens and closes in response to differential pressures. Patients with valvular disease have abnormal anatomy and/or function of at least one valve. For example, a valve may suffer from insufficiency, also referred to as regurgitation, when the valve does not fully close, thereby allowing blood to flow retrograde. Valve stenosis can cause a valve to fail to open properly. Other diseases may also lead to dysfunction of the valves.

The mitral valve, for example, sits between the left atrium and the left ventricle and, when functioning properly, allows blood to flow from the left atrium to the left ventricle while preventing backflow or regurgitation in the reverse direction. Native valve leaflets of a diseased mitral valve, however, do not fully prolapse, causing the patient to experience regurgitation.

While medications may be used to treat diseased native valves, the defective valve often needs to be repaired or replaced at some point during the patient's lifetime. Existing prosthetic valves and surgical repair and/or replacement procedures may have increased risks, limited lifespans, and/or are highly invasive. Some less invasive transcatheter options are available, but most are not ideal. A major limitation of existing transcatheter mitral valve devices, for example, is that the mitral valve devices are too large in diameter to be delivered transseptally, requiring transapical access instead. Furthermore, existing mitral valve replacement devices are not optimized with respect to strength-weight ratio and often take up too much space within the valve chambers, resulting in obstruction of outflow from the ventricle into the aorta and/or thrombosis.

Thus, a new valve device that overcomes some or all of these deficiencies is desired.

### SUMMARY

Described herein is a device for repair and/or replacement of heart valves, including the mitral valve, that is deliverable through minimally invasive techniques and that comprises a minimal amount of valve and/or stent material. Not necessarily all such aspects or advantages are achieved by any particular embodiment. Thus, various embodiments may be realized in a manner that achieves or optimizes one or more advantage or group of advantages taught herein without necessarily achieving other aspects or advantages as may also be taught or suggested herein.

The present disclosure generally relates to prosthetic heart valves for treatment or replacement of a diseased native valve in a patient and more particularly relates to prosthetic heart valves formed from a minimal amount of material and/or having a stiff region of minimal length.

The present disclosure generally relates to treating a diseased native valve in a subject, and more particularly relates to prosthetic heart valves.

In general, in one embodiment, a device for treating a diseased native valve in a patient includes a frame structure and a valve segment. The frame structure has an unexpanded configuration and an expanded configuration. The frame structure in the expanded configuration includes a first end portion, a central annular portion, and a second end portion. The valve segment is coupled to the central annular portion and at least partially longitudinally aligned with the first end portion when the frame structure is in the expanded configuration. The valve segment includes a biocompatible one-way valve. An inflow edge of the valve segment is unsupported by the first end portion of the frame structure and spaced radially inwards from the first end portion when the frame structure is in the expanded configuration.

This and any other embodiments can include one or more of the following features. The frame structure can have a longitudinal length of less than 35 mm in the expanded configuration. The valve segment can include a plurality of leaflets. The valve segment can further include a seal positioned radially between the frame structure and the plurality of leaflets. The seal can be attached to the central annular portion of the frame structure. The plurality of leaflets can be attached to the frame structure only at commissures of the leaflets. The plurality of leaflets can be supported at a nadir of each leaflet by a nadir support extending from the central annular portion. At least portion of the inflow edge can extend beyond the frame structure while an entire outflow edge of the valve segment is positioned within the frame structure. The first end portion can flare radially outwards from the central annular portion. The second end portion can flare radially outwards from the central annular portion. The first and second end portions can be configured to engage an exterior anchor therebetween when the frame structure is in the expanded configuration. The central portion can be configured to apply outward radial pressure to engage with an exterior anchor. The frame structure can be configured to self-expand from the unexpanded configuration to the expanded configuration. The frame structure can be configured to couple to the native valve such that the first end portion is oriented toward an atrial side of the native valve and the second end portion is oriented toward a ventricular side of the native valve. The diameter of the frame structure can be the expanded configuration is between 25 mm and 35 mm.

In general, in one embodiment, a device for treating a diseased native valve in a patient includes a frame structure and a valve segment. The frame structure has an unexpanded configuration and an expanded configuration. The valve segment is coupled to the frame structure. The valve segment includes a biocompatible one-way valve further having an inflow edge and an outflow edge. A majority of the inflow edge of the valve segment is unsupported by the frame structure. An inflow end of the frame structure includes a plurality of flared flanges extending radially therefrom. The frame structure in the expanded configuration includes a first end portion, a central annular portion, and a second end portion. The inflow edge of the valve segment is positioned radially inwards of the plurality of flared flanges when the frame structure is in the expanded configuration.

This and any other embodiments can include one or more of the following features. The frame structure can have a longitudinal length of less than 35 mm in the expanded configuration. The valve segment can include a plurality of leaflets. The valve segment can further include a seal positioned radially between the frame structure and the plurality of leaflets. The seal can be attached to a central portion of the frame structure. The plurality of leaflets can be attached to the frame structure only at commissures of the leaflets. The plurality of leaflets can be supported at a nadir of each leaflet by a nadir support extending from the frame structure. At least portion of the inflow edge can extend beyond the frame structure while an entire outflow edge of the valve segment can be positioned within the frame structure. An outflow end of the frame structure can include a plurality of flared flanges extending radially therefrom. The inflow end and the outflow end can be configured to engage an exterior anchor therebetween when the frame structure is in the expanded configuration. The frame structure can include a central annular portion configured to apply outward radial pressure to engage with an exterior anchor. The frame structure can be configured to self-expand from the unexpanded configuration to the expanded configuration. The frame structure can be configured to couple to the native valve such that the inflow edge is oriented toward an atrial side of the native valve and the outflow edge is oriented toward a ventricular side of the native valve. The diameter of the frame structure in the expanded configuration can be between 25 mm and 35 mm.

In general, in one embodiment, a device for treating a diseased native valve in a patient includes a frame structure and a valve segment. The frame structure has an unexpanded configuration and an expanded configuration. The valve segment is coupled to the frame structure and includes a biocompatible one-way valve having a plurality of leaflets. The valve segment further includes an inflow edge and an outflow edge. The inflow edge of the valve segment is attached to the frame structure only at commissures of the plurality of leaflets.

This and any other embodiments can include one or more of the following features. The frame structure can have a longitudinal length of less than 35 mm in the expanded configuration. The valve segment can include a plurality of leaflets. The valve segment can further include a seal positioned radially between the frame structure and the plurality of leaflets. The seal can be attached to a central portion of the frame structure. The plurality of leaflets can be supported at a nadir of each leaflet by a nadir support extending from the frame structure. At least portion of the inflow edge can extend beyond the frame structure while the entire outflow edge can be positioned within the frame structure. An inflow end of the frame structure can include a plurality of flared flanges extending radially therefrom. The valve segment can be spaced radially inwards from the plurality of flared flanges. An outflow end of the frame structure can include a plurality of flared flanges extending radially therefrom. The inflow end and the outflow end can be configured to engage an exterior anchor therebetween when the frame structure is in the expanded configuration. The frame structure can include a central annular portion configured to apply outward radial pressure to engage with an exterior anchor. The frame structure can be configured to self-expand from the unexpanded configuration to the expanded configuration. The frame structure can be configured to couple to the native valve such that the inflow edge is oriented toward an atrial side of the native valve and the outflow edge is oriented toward a ventricular side of the native valve. The diameter of the frame structure in the expanded configuration can be between 25 mm and 35 mm.

In general, in one embodiment, device for treating a diseased native valve in a patient includes a frame structure and a valve segment. The frame structure has an unexpanded configuration and an expanded configuration. The frame structure includes a first end portion, a second end portion, and a central annular portion. The valve segment is coupled to the frame structure and includes a biocompatible one-way valve. The frame structure includes a plurality of expandable cells. The ratio of a height of the frame structure to a diameter of the frame structure when the frame structure is in the expanded configuration is from 0.05 to 0.65.

This and any other embodiments can include one or more of the following features. The frame structure can have a longitudinal length of less than 35 mm in the expanded configuration. The valve segment can include a plurality of leaflets. The valve segment can further include a seal positioned radially between the frame structure and the plurality of leaflets. The seal can be attached to the central annular portion of the frame structure. The plurality of leaflets can be attached to the frame structure only at commissures of the leaflets. The plurality of leaflets can be supported at a nadir of each leaflet by a nadir support extending from the central annular portion. At least portion of the valve segment can extend beyond the first end portion while an entire outflow edge of the valve segment can be positioned between the first end portion and the second end portion. The first end portion can include a plurality of flared flanges extending radially therefrom. The second end portion can include a plurality of flared flanges extending radially therefrom. The first end portion and the second end portion can be configured to engage an exterior anchor therebetween when the frame structure is in the expanded configuration. The central annular portion can be configured to apply outward radial pressure to engage with an exterior anchor. The frame structure can be configured to self-expand from the unexpanded configuration to the expanded configuration. The frame structure can be configured to couple to the native valve such that the first end portion is oriented toward an atrial side of the native valve and the second end portion is oriented toward a ventricular side of the native valve. The diameter of the frame structure in the expanded configuration can be between 25 mm and 35 mm.

In general, in one embodiment, a method for treating a diseased native mitral valve of a subject includes advancing a distal end of a delivery device from a first side of the native mitral valve to a second side of the native mitral valve, advancing a frame structure with a valve segment therein from the delivery device, and expanding the frame structure within the native valve from an unexpanded configuration to an expanded configuration such that a cylindrical annular portion of the frame structure engages with an anchor positioned around chordae of the native valve and such that the valve segment is unsupported by the frame structure within an atrium of the subject.

This and any other embodiments can include one or more of the following features. Expanding the frame structure can further allow the valve segment to partially collapse radially outwards during diastole. Expanding the frame structure can further allow the valve segment to billow inwards so as to block the flow of blood therethrough during systole. The billowing can occur in the unsupported portion of the valve segment. The unsupported portion can include a seal positioned along an inner circumference of the frame structure and leaflets attached to the seal. The frame structure can include a first and second opposite ends. Expanding the frame structure can include expanding the frame structure such that the first end extends above the first side of the native valve and the second end extends below the second side of the native valve. The method can further include delivering an anchor around chordae of the native valve. Expanding the frame structure can include expanding the frame structure such that it engages with the anchor. The frame structure can be self-expanding. Expanding the frame structure can include releasing the frame structure from radial constriction by the delivery device. The method can further include transseptally inserting the distal end of the delivery device in to a left atrium of the heart of the subject.

An aspect of the present disclosure provides a device for treating a diseased native valve in a patient. The device comprises a frame structure and a valve segment. The frame structure has an unexpanded configuration and an expanded configuration. The frame structure comprises a first portion and a second portion. The first portion of the frame structure is coupled to the valve segment so that the valve segment functions as a prosthetic valve when the frame structure is in the expanded configuration within the native valve. The first portion of the frame structure comprises a plurality of expandable cells. The second portion of the frame structure is configured to secure the frame structure in a longitudinal position relative to the native valve.

In some embodiments, a diameter of the frame structure is no more than 15 mm when the frame structure is in the unexpanded configuration.

In some embodiments, the second portion of the frame structure is rigid. In some embodiments, the second portion of the frame structure has a longitudinal length of less than 35 mm.

In some embodiments, the frame structure comprises a plurality of struts defining the plurality of expandable cells. In some embodiments, the plurality of struts comprises a lattice structure. In some embodiments, the lattice structure comprises at least a portion of the first portion of the frame structure.

In some embodiments, one or more expandable cells of the plurality of expandable cells are diamond-shaped when the frame structure is in the expanded configuration.

In some embodiments, at least a portion of the valve segment is positioned at the same longitudinal location as at least a portion of the frame structure when the frame structure is in the expanded configuration.

In some embodiments, the first portion of the frame structure is disposed in a longitudinal location nearer to a first end of the device than the second portion of the frame structure when the frame structure is in the unexpanded configuration.

In some embodiments, the valve segment comprises at least one leaflet having an inner layer and an outer layer. In some embodiments, the frame structure is attached to the outer layer at one or more longitudinal ends of the frame structure. In some embodiments, the valve segment comprises a plurality of leaflets.

In some embodiments, the second portion of the frame structure is configured to affix to the native valve with the aid of an outer anchor.

In some embodiments, the outer anchor is configured to couple to the frame structure by positioning native tissue between the frame structure and the outer anchor to affix the frame structure to the anchor.

In some embodiments, the frame structure comprises one or more coil grabbers.

In some embodiments, the frame structure comprises a plurality of hoop structures. In some cases, at least one hoop structure of the plurality of hoop structures is connected to a leaflet of the plurality of leaflets. In some embodiments, the second portion of the frame structure comprises three or more hoop structures.

In some embodiments, the second portion of the frame structure comprises a first longitudinal end and a second longitudinal end.

In some embodiments, the first portion of the frame structure comprises at least one flange. In some embodiments, the at least one flange is coupled to the frame structure at the first longitudinal end of the second portion of the frame structure. In some embodiments, one or more flanges of the at least one flange are nested flanges.

In some embodiments, the frame structure is balloon-expandable.

In some embodiments, the frame structure is sized to be positioned in the mitral valve.

In some embodiments, the device further comprises a fabric covering coupled to the frame structure.

In some embodiments, the frame structure is configured to couple to the native valve such that the first portion is oriented toward an atrial side of the native valve and the second portion is oriented toward a ventricular side of the native valve.

In another aspect, a system for the delivery of a prosthetic device is provided. The system comprises the device for treating a diseased native valve in a patient and a delivery device comprising an outer sheath.

In another aspect, a device for treating a diseased native valve in a patient is presented. The device comprises a frame structure and a valve segment coupled to the frame structure. The valve segment comprises a biocompatible one-way valve. The frame structure has an unexpanded configuration and an expanded configuration. The frame structure comprises a first portion and a second portion. The second portion of the frame structure is coupled to the valve segment so that the valve segment functions as a prosthetic valve when the frame structure is in the expanded configuration within the native valve. The first portion of the frame structure comprises at least three flanges coupled to the frame structure. The second portion comprises a plurality of expandable cells.

In some embodiments, a diameter of the frame structure is no more than 15 mm when the frame structure is in the unexpanded configuration.

In some embodiments, one or more flanges of the at least three flanges are coupled to a first end of the second portion of the frame structure. In some embodiments, the one or more flanges of the at least three flanges are coupled to one or more arches at the first end of the second portion of the frame structure.

In some embodiments, the one or more flanges of the at least three flanges form a flange angle with the frame structure, the flange angle being between 0 degrees and 60 degrees.

In some embodiments, the at least three flanges comprise one or more nested flanges. In some embodiments, a nested flange of the one or more nested flanges comprises at least two flange tips. In some embodiments, the radial length of one or more flanges of the at least three flanges is from 1 mm to 5 mm.

In some embodiments, the device further comprises a plurality of coil grabbers. In some embodiments, the plurality of coil grabbers are coupled to the second end of the second portion of the frame structure.

In some embodiments, the first portion of the frame structure has a longitudinal length of no more than 35 mm. In some embodiments, the diameter of the frame structure in the expanded configuration is from 25 to 35 mm.

In some embodiments, one or more expandable cell of the plurality of expandable cells is diamond-shaped when the frame structure is in the expanded configuration.

In some embodiments, the device further comprises an anchor coupled to the second end of the second portion of the frame structure.

In some embodiments, circumferentially adjacent flanges are disposed at a circumferential angle with respect to one another, the circumferential angle being from 10 degrees and 180 degrees, from 20 degrees to 150 degrees, from 30 degrees to 120 degrees, from 45 degrees to 90 degrees, or 60 degrees to 72 degrees.

In another aspect, a device for treating a diseased native valve in a patient is presented. The device comprises a frame structure and a valve segment coupled to the frame structure. The valve segment comprises a biocompatible one-way valve. The frame structure has an unexpanded configuration and an expanded configuration. The frame structure comprises a first portion and a second portion. The second portion of the frame structure is coupled to the valve segment so that the valve segment functions as a prosthetic valve when the frame structure is in the expanded configuration within the native valve. The first portion of the frame structure comprises at least five flanges coupled to the frame structure. The second portion of the frame structure comprises a plurality of expandable cells.

In some embodiments, a diameter of the frame structure is no more than 15 mm when the frame structure is in the unexpanded configuration.

In some embodiments, one or more flanges of the at least five flanges are coupled to a first end of the second portion of the frame structure.

In some embodiments, the one or more flanges of the at least five flanges are coupled to one or more arches at the first end of the second portion of the frame structure.

In some embodiments, the one or more flanges of the at least five flanges forms a flange angle with the frame structure, the flange angle being between 0 degrees and 60 degrees.

In some embodiments, the at least five flanges comprise one or more nested flanges. In some embodiments, a nested flange of the one or more nested flanges comprises at least two flange tips. In some embodiments, the radial length of one or more flanges of the at least five flanges is from 1 mm to 5 mm.

In some embodiments, the device further comprises a plurality of coil grabbers coupled to the second end of the second portion of the frame structure.

In some embodiments, the first portion of the frame structure has a longitudinal length of no more than 35 mm.

In some embodiments, one or more expandable cells of the plurality of expandable cells are diamond-shaped when the frame structure is in the expanded configuration.

In some embodiments, the device further comprises an anchor coupled to the second end of the second portion of the frame structure.

In some embodiments, circumferentially adjacent flanges are disposed at a circumferential angle with respect to one another. In some embodiments, the circumferential angle is from 10 degrees to 180 degrees, from 20 degrees to 150 degrees, from 30 degrees to 120 degrees, from 45 degrees to 90 degrees, or from 60 degrees to 72 degrees.

In another aspect, a method for treating a diseased native valve in a subject is presented. The method comprises advancing a distal end of a delivery device from a first side of a native valve to a second side of the native valve. The delivery device comprises an outer sheath comprising a lumen and an opening. The distal end of the delivery device is detachably coupled to a prosthetic valve device. The prosthetic valve device comprises a valve segment and a frame structure. The valve segment is coupled to the frame structure. The method comprises advancing the frame structure from the delivery device. The method comprises expanding the frame structure within the native valve from an unexpanded configuration to an expanded configuration. The method comprises, after advancing the frame structure, deploying an anchor from the delivery device to couple to the expanded frame structure.

In some embodiments, the method further comprises releasing the prosthetic valve device from the distal end of the delivery device. In some embodiments, the delivery device is configured to deliver the prosthetic valve from a portion of the delivery device proximal to the distal end of the delivery device.

In some embodiments, the method further comprises retracting the delivery device from the native valve.

In some embodiments, the frame structure has a diameter of no more than 15 mm when the frame structure is in the unexpanded configuration.

In some embodiments, the frame structure comprises a first and second opposite ends. In some embodiments, expanding the frame structure comprises expanding the frame structure such that the first end extends above the first side of the native valve and the second end extends below the second side of the native valve.

In some embodiments, deploying the anchor comprises advancing the anchor through the second end of the frame structure. In some embodiments, the anchor is configured to wrap at least partially around the frame structure as it is deployed.

In some embodiments, the method further comprises wrapping the anchor around one or more structures on the second side of the native valve. In some embodiments, the one or more structures comprise one or more valve leaflets of the native valve. In some embodiments, the one or more structures comprise one or more chordae of the left ventricle.

In some embodiments, the frame structure further comprises one or more flanges coupled to the first end of the frame structure. In some embodiments, the frame structure comprises at least three flanges coupled to the first end of the frame structure. In some embodiments, the frame structure comprises at least five flanges coupled to the first end of the frame structure.

In some embodiments, expanding the frame structure comprises inflating a balloon disposed within the frame structure.

In some embodiments, the frame structure is self-expanding. In some embodiments, expanding the frame structure comprises releasing the frame structure from radial constriction by a delivery device (e.g., in a system comprising valve prosthesis 10 and a delivery device comprising an outer sheath). For example, a self-expanding valve prosthesis 10 can be expanded by releasing the valve prosthesis 10 from a distal end of a delivery device (or a location proximal to the distal end of the delivery device).

In some embodiments, the native valve is in a heart of the subject. In some embodiments, the method further comprises transeptally inserting the distal end of the delivery device into a left atrium of the heart of the subject. In some embodiments, the native valve comprises a mitral valve. In some embodiments, the first side of the native valve comprises a left atrium. In some cases the second side of the native valve comprises a left ventricle.

In another aspect, a method of treating a diseased heart valve is presented. The method comprises introducing a device according to any one of the embodiments described herein to a valve to be treated. The method comprises using the device to treat the valve. In some embodiments, the using comprises repairing the diseased valve. In some embodiments, the using comprises replacing the diseased valve.

In another aspect, a method for treating a diseased native valve in a patient is provided. The method comprises advancing a distal end of a delivery device from a first side of a native valve to a second side of the native valve. The delivery device comprises an outer sheath comprising a lumen and an opening. The distal end of the delivery device is detachably coupled to a prosthetic valve device. The prosthetic valve device comprises an anchor, a valve segment, and a frame structure. The anchor and valve segment are coupled to the frame structure. The method comprises expanding the frame structure within the native valve from an unexpanded configuration to an expanded configuration. The method comprises releasing the prosthetic valve device from the distal end of the delivery device. The method comprises retracting the delivery device from the native valve.

In some embodiments, the anchor is configured to wrap at least partially around the frame structure in the deployed configuration.

In some embodiments, advancing the anchor comprises pushing the anchor through the native valve. In some embodiments, advancing the anchor further comprises rotating the anchor.

In some embodiments, the frame structure comprises a first and second opposite ends. In some embodiments, expanding the frame structure comprises expanding the frame structure such that the first end extends above the first side of the native valve and the second end extends below the second side of the native valve.

In some embodiments, expanding the frame structure comprises expanding at least a portion of the frame structure within at least a portion of the deployed anchor to anchor the frame structure to the native valve.

In some embodiments, expanding the frame structure and releasing the frame structure occur simultaneously.

In some embodiments, the frame structure is balloon-expandable. In some embodiments, expanding the frame structure comprises inflating a balloon disposed within the frame structure. In some embodiments, inflation of the balloon causes expansion of the frame structure.

In some embodiments, the frame structure is self-expanding. In some embodiments, expanding the frame structure comprises releasing the frame structure from radial constriction by the delivery device.

In some embodiments, the method further comprises rotating a free end of the deployed anchor around one or more structures on the second side of the native valve. In some embodiments, the one or more structures comprise one or more valve leaflets of the native valve. In the embodiments, one or more structures comprise one or more chordae of the left ventricle.

In some embodiments, the valve segment comprises a biocompatible one-way valve.

In some embodiments, the native valve is in a heart of a subject. In some embodiments, the method further comprises transseptally inserting the distal end of the delivery device into a left atrium of the heart of the subject. In some embodiments, the native valve comprises a mitral valve. In some embodiments, the first side of the native valve comprises a left atrium. In some embodiments, the second side of the native valve comprises a left ventricle.

In another aspect, a device for treating a diseased native valve in a patient is provided. T he device comprises a frame structure having an unexpanded configuration and an expanded configuration, the frame structure comprising a first portion and a second portion, and a valve segment coupled to the frame structure. The valve segment comprises a biocompatible one-way valve. The second portion of the frame structure is coupled to the valve segment so that the valve segment functions as a prosthetic valve when the frame structure is in the expanded configuration within the native valve. The second portion of the frame structure comprises a plurality of expandable cells. The ratio of a height of the frame structure to a diameter of the frame structure when the device is in the expanded configuration is from 0.05 to 0.65.

In some embodiments, the valve segment is coupled to the frame structure at a distal end of the device. For example, in some embodiments, the valve segment is coupled continuously around the circumference of the device. Alternatively, or in combination, in some embodiments the valve segment is coupled to a distal arch of the device.

In some embodiments, at least a portion of the valve segment comprises a biological tissue.

In some embodiments, at least a portion of the valve segment comprises decellularized pericardium.

These and other embodiments are described in further detail in the following description related to the appended drawing figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1 shows a perspective view of an implantable valve prosthesis, in accordance with embodiments.

FIG. 2 shows a side view of the implantable valve prosthesis of FIG. 1 crimped, in accordance with embodiments.

FIG. 3 shows a side view of the implantable valve prosthesis device of FIG. 1 connected to an anchor, in accordance with embodiments.

FIG. 4 shows a side view of an implantable valve prosthesis comprising three flanges with an anchor, in accordance with embodiments.

FIG. 5 shows a top view of the implantable valve prosthesis of FIG. 4, in accordance with embodiments.

FIG. 6 shows a bottom view of the implantable valve prosthesis of FIG. 4, in accordance with embodiments.

FIG. 7 shows a perspective view of the implantable valve prosthesis of FIG. 4, in accordance with embodiments.

FIG. 8 shows a perspective view of the frame structure of an implantable valve prosthesis comprising five flanges, in accordance with embodiments.

FIG. 9 shows a perspective view of an implantable valve prosthesis of FIG. 8 with an anchor, in accordance with embodiments.

FIG. 10 shows a top view of the implantable valve prosthesis with anchor of FIG. 9, in accordance with embodiments.

FIG. 11 shows a bottom view of the implantable valve prosthesis with anchor of FIG. 9, in accordance with embodiments.

FIG. 12 shows a side view of the implantable valve prosthesis with anchor of FIG. 9, in accordance with embodiments.

FIG. 13 shows a perspective view of an implantable valve prosthesis, in accordance with embodiments.

FIG. 14 shows a perspective view of an implantable valve prosthesis, in accordance with embodiments.

FIG. 15 shows a perspective view of an implantable valve prosthesis, in accordance with embodiments.

FIG. 16A shows a side view of an implantable valve prosthesis with the valve segment extending proximal of the strut frame, in accordance with embodiments.

FIG. 16B shows a bottom view (i.e., from the outflow end) of the implantable valve prosthesis of FIG. 16A.

FIG. 17A shows a portion of a valve prosthesis, in accordance with embodiments.

FIG. 17B shows a bottom view of the valve prosthesis of FIG. 17A.

FIG. 17C shows a detailed side view of the valve prosthesis of FIG. 17A.

FIG. 18A shows a side view of a valve prosthesis, in accordance with embodiments.

FIG. 18B shows a bottom view of the valve prosthesis of FIG. 18A.

FIG. 19A shows a side view of an implantable prosthesis with minimal valve supports, in accordance with embodiments.

FIG. 19B shows a bottom view of the prosthesis of FIG. 19A.

FIG. 20 shows a perspective view of a valve prosthesis, in accordance with embodiments.

FIG. 21 shows a detailed side view of a valve prosthesis with minimal valve supports, in accordance with embodiments.

FIG. 22 shows a detailed side view of a valve prosthesis with minimal valve supports, in accordance with embodiments.

FIG. 23 shows a detailed side view of a valve prosthesis with minimal valve supports, in accordance with embodiments.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments, however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The present disclosure is described in relation to systems, devices, or methods for treatment or replacement of a diseased native valve of the heart, for example a mitral valve. However, one of skill in the art will appreciate that this is not intended to be limiting and the devices and methods disclosed herein may be used in other anatomical areas and in other surgical procedures.

An exemplary valve prosthesis 10 as described herein can include a frame structure 12 and a valve segment 14 positioned therein. Valve segment 14 can comprise a plurality of valve leaflets 16. In an expanded configuration, valve segment 14 can function as a fluidic valve in place of a native valve tissue (e.g., a heart valve, such as the mitral valve). The frame structure 12 can provide circumferential strength and/or longitudinal strength to valve prosthesis device 10.

One or more portions of valve prosthesis 10 can be shaped or configured to aid in securing valve prosthesis 10 at a location (e.g., in the orifice of a native heart valve). Described herein, for example, are various embodiments of anchors (e.g., spiral anchors 15) and flared portions (e.g., with flanges 159) that can aid in establishing or maintaining the valve prosthesis 10 at a location. In some embodiments, the valve prosthesis 10 can comprise one or more hook, barb, or scallop-shaped anchor to aid in deployment and/or positioning of valve prosthesis 10 at a location. In some cases, one or more hooks, barbs, or scallop-shaped anchor may be coupled to a portion of frame structure 12 (e.g., at a commissural post 117, a strut 113, a proximal arch 115, or a distal arch 116). For example, the frame structure 12 may comprise one or more hooks or barbs (e.g., connected to a strut 113), which can contact a tissue of a native heart valve or a tissue surrounding a native heart valve to prevent valve prosthesis 10 from moving or becoming dislodged from a location at which it has been placed or deployed.

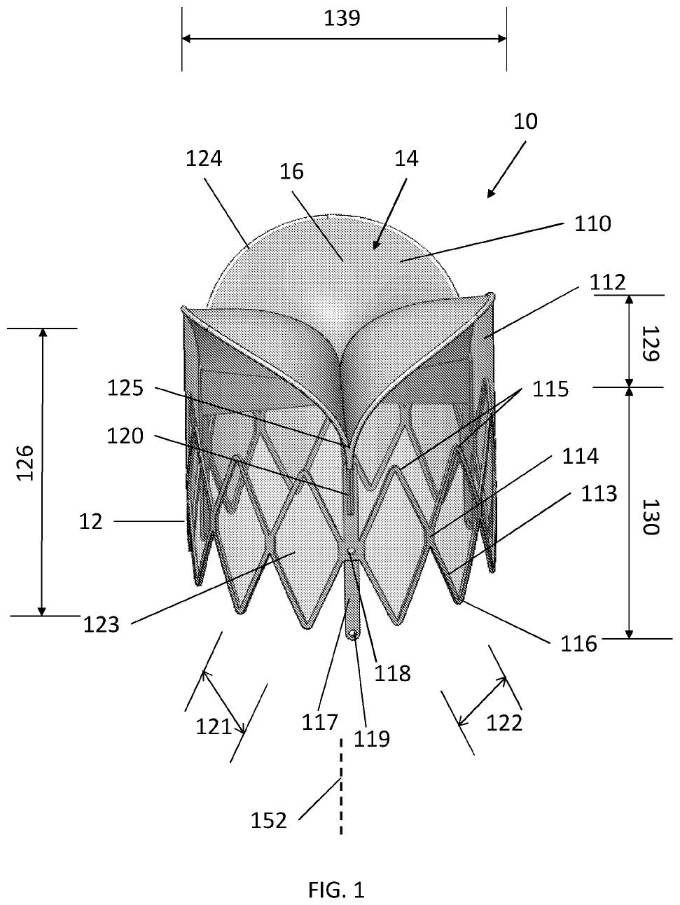

FIG. 1 shows a valve prosthesis 10 (e.g., an implantable valve prosthesis) in an expanded configuration. The valve prosthesis 10 can be deployed in an expanded configuration according to the methods described herein. For example, valve prosthesis 10 can be deployed into an expanded configuration in a method of replacing or repairing. In the expanded configuration, valve prosthesis 10 can be positioned and/or anchored at a target region of a subject (e.g., an organ or tissue of an animal such as a dog, cat, horse, or human). For example, valve prosthesis 10 can be positioned in the expanded configuration in the orifice of a heart valve, such as the mitral valve or tricuspid valve (e.g., to function as a temporary or permanent replacement for an existing mitral valve or tricuspid valve of the heart).

Various methods and mechanisms can be used to place the valve prosthesis 10 in the expanded configuration.

In one embodiment, the valve prosthesis 10 may be balloon-expandable, self-expanding, or otherwise expansible as will be understood to one of ordinary skill in the art based on the teachings herein. For example, a delivery system may comprise an expandable member, such as an inflatable balloon or expandable malecot, e.g., disposed within the valve prosthesis device 10. Inflation of the balloon or, more generally, expansion of the expandable member, may cause expansion of the valve prosthesis device 10. Valve prosthesis device 10, or at least one element thereof (e.g., frame structure 12), can be balloon-expandable. For example, a balloon catheter can be threaded through a longitudinal axis of the valve prosthesis 10 in an unexpanded (or partially expanded) configuration.

In another embodiment, valve prosthesis 10, or at least one element thereof (e.g., at least a portion of the frame structure 12), can be self-expanding. The valve prosthesis 10 may be maintained in the unexpanded configuration, for example, by radial constriction from an outer sheath of the delivery device when disposed in a lumen of the outer sheath. Advancement of an inner shaft or tether of the delivery system distally and/or out of the outer sheath may actuate the valve prosthesis 10 into the expanded configuration. In some cases, retraction of the outer sheath away from the valve prosthesis 10 may actuate the valve prosthesis 10 into the expanded configuration.

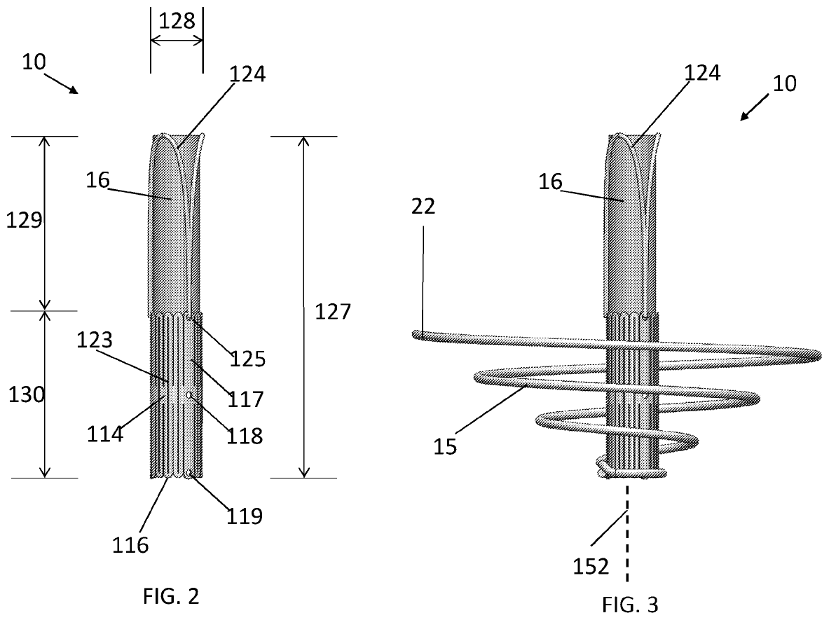

FIG. 2 shows the valve prosthesis 10 in an unexpanded (or collapsed or crimped) configuration. In some cases, the valve prosthesis 10 can be delivered to a target region (e.g., a region of a heart comprising a native valve) in the unexpanded configuration. In some cases, the valve prosthesis 10 in the unexpanded configuration can allow the valve prosthesis 10 to be delivered via minimally invasive means (e.g., via a delivery device, as described herein).

Referring to FIG. 2, the longitudinal length 127 of the collapsed valve prosthesis 10 can be minimized, which can be advantageous for delivery of the valve prosthesis 10. For example, minimizing the overall longitudinal length 127 of the collapsed valve prosthesis 10 can allow improved maneuverability within a delivery device while maintaining structural strength of the device. In some cases, minimizing the overall longitudinal length 127 of the collapsed valve prosthesis 10 can allow insertion of valve prosthesis 10 through an access path that would be challenging for a longer device to traverse (e.g., an access path comprising tortuous passages or passages with sharp turns). In some cases, the valve prosthesis 10 in the unexpanded configuration has an overall longitudinal length 127 of from 1 mm to 50 mm, from 1 mm to 45 mm, from 1 mm to 40 mm, from 1 mm to 35 mm, from 1 mm to 30 mm, from 1 mm to 25 mm, from 1 mm to 20 mm, from 1 mm to 10 mm, from 10 mm to 45 mm, from 20 mm to 45 mm, from 20 mm to 30 mm, from 25 mm to 35 mm, or from 27.5 mm to 32.5 mm. In some cases, the prosthetic delivery device 10 in the expanded In some cases, the prosthetic delivery device 10 in the expanded configuration can have an overall longitudinal length of from 1 mm to 45 mm, from 10 mm to 45 mm, from 15 mm to 45 mm, from 15 mm to 35 mm, from 16 mm to 34 mm, from 17 mm to 33 mm, from 18 mm to 32 mm, from 19 mm to 31 mm, from 20 mm to 30 mm, from 25 mm to 35 mm, or from 27.5 mm to 32.5 mm. In some embodiments, the valve prosthesis 10 can foreshorten as it expands such that the length 126 in the expanded configuration is less than the length 127 in the collapsed configuration.

Further, the diameter 128 of the collapsed valve prosthesis 10 can be minimized, which can likewise be advantageous for delivery of the valve prosthesis 10. For example, a collapsed valve prosthesis 10 with a smaller diameter 128 can fit inside of a delivery device with a smaller diameter, allowing for less invasive delivery and for improved maneuvering capability inside of a subject's body. Reducing the diameter 128 of the collapsed valve prosthesis 10 (e.g., for use in treatment or replacement of a mitral valve, a tricuspid valve, an aortic valve, or a pulmonic valve) can further allow for easier delivery of the valve prosthesis 10 to a target region of a subject, faster recovery of a subject receiving valve prosthesis 10, and/or improved clinical outcomes for a subject receiving valve prosthesis 10 (e.g., improved subject survival, improved ejection fraction, improved cardiac output, decreased valvular regurgitation, and/or decreased edema). In some cases, reducing the diameter 128 of the collapsed valve prosthesis 10 can make transseptal access and delivery possible in addition to transapical access. In some cases, the diameter 128 of the collapsed valve prosthesis 10 or portion thereof (e.g., frame structure 12) can be from 0.01 mm to 20 mm, 0.01 mm to 15 mm, 0.01 mm to 10 mm, from 0.01 mm to 9 mm, from 0.01 mm to 8 mm, from 0.01 mm to 7 mm, from 0.01 mm to 6 mm, from 0.01 mm to 5 mm, from 0.01 mm to 4 mm, from 0.01 mm to 3 mm, from 0.01 mm to 2 mm, from 0.01 mm to 1 mm, from 1 mm to 15 mm, from 2 mm to 14 mm, from 3 mm to 13 mm, from 4 mm to 12 mm, from 5 mm to 10 mm, from 6 mm to 10 mm, from 7 mm to 10 mm, from 8 mm to 10 mm, from 9 mm to 10 mm, from 10 mm to 15 mm, no more than 20 mm, no more than 15 mm, no more than 10 mm, no more than 9 mm, no more than 8 mm, no more than 7 mm, no more than 6 mm, or no more than 5 mm.

The diameter 139 of frame structure 12 in an expanded configuration (see FIG. 1) can be larger than the diameter 128 of frame structure 12 in an unexpanded configuration (see FIG. 2). In some cases, frame structure 12 or a portion thereof (e.g., annular central portion 158 of frame structure 12) can have an expanded diameter 139 of from 10 mm to 50 mm, from 20 mm to 40 mm, from 25 mm to 35 mm, from 27 mm to 33 mm, no more than 50 mm, no more than 40 mm, no more than 35 mm, no more than 33 mm, no more than 30 mm, no more than 25 mm, no more than 20 mm, or no more than 15 mm when frame structure 12 is in an expanded configuration.

In some cases, the diameter 128 or 139 refers to a largest cross-sectional width of valve prosthesis 10 or a portion thereof, e.g., as measured in a plane perpendicular to a longitudinal axis of the valve prosthesis 10 at a longitudinal location. In some situations, the valve prosthesis 10 has a polygonal cross-section. In some cases, the diameter 128, 139 can refer to the largest distance from a first side of a polygonal cross-section of the valve prosthesis 10 to a second side of the polygonal cross-section of the valve prosthesis 10.

In some cases, the valve prosthesis 10 or a portion thereof can be sized or shaped to be positioned at a certain location or target region. For example, the frame structure 12 can be sized to be positioned in a valve, such as the mitral valve (e.g., by designing a dimension of frame structure to fit a valve, such as the mitral valve, when in an expanded configuration).

As shown in FIGS. 1-2, the valve prosthesis 10 can include a first portion 129 comprising only the valve segment 14 and/or minimal valve supports 124 and a second portion 130 comprising the frame structure 12 and the valve segment 14. In some embodiments, the valve segment 14 can be entirely unsupported or mostly unsupported in the first portion 129 while the valve segment 14 can be completely supported in the second portion 130 (e.g., by the frame structure 12). For example, minimal valve supports 124 can extend from the frame structure 12 to support the valve segment 14 in the first portion 129. The minimal valve supports 124 can, for example, support only the inflow edges of the valve segment 14 in the first portion 129 while leaving the rest of the valve segment 14 unsupported in the first portion 129. The first portion 129 of the valve prosthesis 10 can be coupled to or continuous with the second portion 130. For example, the frame structure 12 can be coupled to the minimal valve supports 124 at a joint 125 (e.g., with a fastener or crimp) or can be continuous with the minimal valve supports 124 (e.g., via fusion, welding, or formation by a continuous piece of material). Further, the valve segment 14 can be coupled to the minimal valve supports 124 in the first portion 129 and to the frame structure 12 in the section portion 130. When, for example, the valve prosthesis 10 is deployed in an orifice of the native mitral valve, the valve prosthesis 10 can be oriented such that the first portion 129 is positioned closer to the atrium than the second portion 130, and the second portion 130 can be positioned closer to the ventricle of the heart than the first portion 129.

FIG. 3 shows a representative example of the valve prosthesis 10 in an unexpanded configuration coupled to an anchor 15. In some embodiments, the anchor 15 may comprise a spiral shape that, for example, spirals around the valve prosthesis 10 in the unexpanded and/or expanded configuration. The anchor 15 can have a free end 22. In some cases, the free end 22 of anchor 15 can be useful during deployment of the anchor 15 in a native heart valve (e.g., by ensnaring chordae or other structures when the prosthesis 10, anchor 15, and/or delivery device are rotated around longitudinal axis of the valve prosthesis 10). The anchor 15 may be directly coupled to the frame structure 12, for example at a first end (e.g., a proximal end) or a second end (e.g., a distal end) thereof. Alternatively, the anchor 15 can be physically uncoupled from the frame structure 12 while providing an anchor for the frame 12 as the frame expands within the native valve orifice (thereby sandwiching tissue between the frame 12 and the anchor 15). In some embodiments, the frame structure 12 can be at least partially held in place within the native valve via interaction with the anchor 15. For example, the expanded diameter of the frame structure 12 can be greater than or equal to the inner diameter of the spiraled anchor 15 such that the frame structure 12 expands into and engages with the anchor 15 (with native valve leaflets, chordae, or other tissue therebetween).

A longitudinal axis of the anchor 15 may be co-axial or concentric with a longitudinal axis of the delivery device when the anchor 15 is in the deployed configuration. In some embodiments, the deployed anchor 15 may be detachably coupled to a delivery device prior to deployment of the valve prosthesis 10. For example, the anchor 15 can be deployed from a delivery device and held with a tether until the frame structure 12 is expanded within the native valve orifice and the anchor 15.

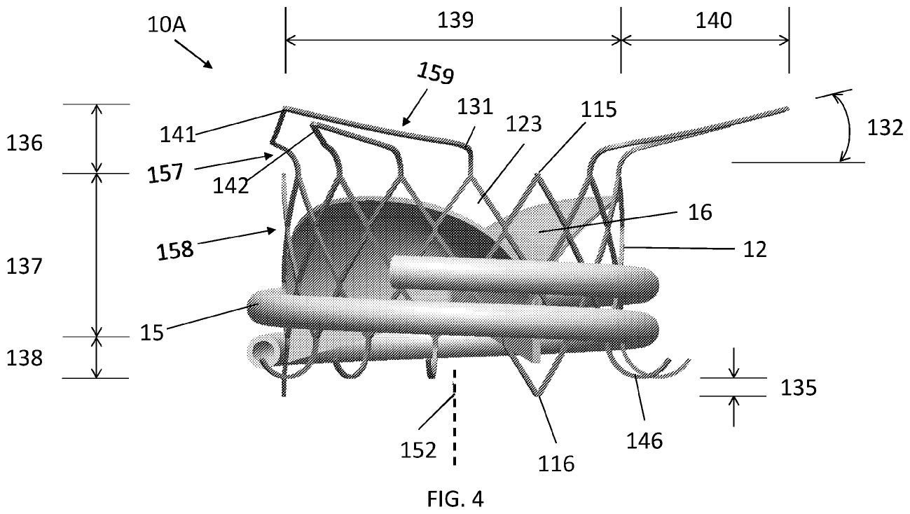

In some embodiments, the valve prostheses 10 described herein can include one or more flared portions to engage with the anchor 15 and/or help prevent the valve prostheses 10 from sliding through a valve orifice. For example, as shown in FIGS. 4-7, the frame structure 12 of valve prosthesis 10A can include an atrial flared portion 157 extending radially outwards from a central annular portion 158. The atrial flared portion 157 can, for example, extend into the atrium of the heart from the central annular portion 158 when valve prosthesis 10A is deployed in a native mitral valve. Alternatively, or in combination, the atrial flared portion 157 can contact a tissue of the atrium of the heart, e.g., a mitral valve annulus when valve prosthesis 10A is deployed in a native mitral valve.

In some embodiments, the atrial flared portion 157 can include flanges 159 (e.g., three discrete flanges 159 as shown in FIGS. 4-7). In some embodiments, the flanges 159 can be coupled to and/or continuous with the frame structure 12. For example, the flanges 159 can extend from one or more proximal arches 115 of the central annular portion 158. The flanges 159 can be, for example, nested flanges including an outer flange tip 141 and an inner flange tip 142 nested within the outer flange tip 141. Each flange tip 141, 142 can be pointed, as shown in FIG. 4, or rounded, or blunt-ended. Each flange 159 can be coupled to frame structure 12 by flange bend 131. The angle of flange bend 131 relative to a cross-sectional plane that is perpendicular to longitudinal axis 152 of the prosthesis 10A (e.g., flange angle 132 as shown in FIG. 4) can be tuned to provide optimal force against the annulus of the native valve without damaging the surrounding tissue. Flange angle 132 can be from 0 degrees to 89 degrees, from 0 degrees to 60 degrees, from 0 degrees to 45 degrees, from 0 degrees to 30 degrees, from 0 degrees to 20 degrees, from 0 degrees to 10 degrees, from 10 degrees to 80 degrees, from 20 degrees to 70 degrees, from 30 degrees to 60 degrees, from 40 degrees to 50 degrees, or from 30 degrees to 45 degrees. In some cases, a flange 159 can extend a distance 136 in a longitudinal direction from the end of the frame structure 12 to which it is coupled. In some cases distance 136 can be from 0 mm to 20 mm, from 1 mm to 10 mm, from 2 mm to 9 mm, from 3 mm to 8 mm, from 4 mm to 7 mm, or from 5 mm to 6 mm.

Referring to FIG. 6, in some cases, the ratio of expanded frame structure diameter 139 to flange length 140 can be from 1:1 to 2:1, from 1:1 to 5:1, from 1:1 to 10:1, from 1:1 to 100:1, from 2:1 to 10:1, from 2:1 to 5:1, or from 3:1 to 5:1. In various embodiments, flange length 140 can be a radial length from flange bend 131 to flange tip 141 or 142. In some cases, flange length 140 can be from 0.1 mm to 8 mm, from 0.5 mm to 7 mm, from 1 mm to 6 mm, from 2 mm to 5 mm, from 3 mm to 4 mm. Further, a first leaflet 16 if valve prosthesis 10A can contact a second leaflet 16 at leaflet contact surface 144. The circumferential angle 145 between a contact surface 144 of the leaflets 16 and a flange can be from: 36 degrees to 60 degrees, from 45 degrees to 55 degrees, from 40 degrees to 60 degrees, from 35 to 65 degrees, from 30 degrees to 70 degrees, from 25 degrees to 75 degrees, from 20 degrees to 80 degrees, from 15 degrees to 85 degrees or from 0 degrees to 90 degrees.

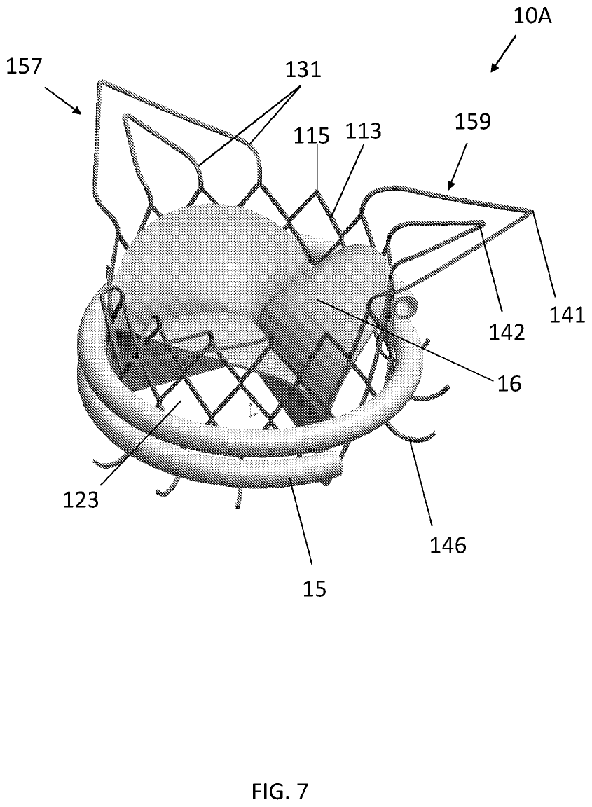

In some embodiments, the valve prostheses 10 described herein can include one or more hooks (or coil/anchor grabbers) projecting radially and/or outwards relative to an exterior of the frame structure 12. For example, prosthesis 10A includes hooks 146 extending from the ventricular/outflow end of the frame structure 12. The hooks 146 extend radially and point back towards the atrial/inflow end of the frame structure 12. The hooks 146 can advantageously prevent anchor 15 from slipping off of an end (e.g., the ventricular end) of frame structure 12 when the prosthesis 10A is implanted in the mitral valve. In some embodiments, the hooks 146 can be coupled directly to a strut (e.g., strut 113 or commissural post 117) or arch (e.g., a distal arch 116) of frame structure 12. The hooks 146 can be equally distributed around the cross-sectional circumference of valve prosthesis 10 or unevenly distributed around the cross-sectional circumference of valve prosthesis 10. In some cases, groups of 2, 3, 4, 5, 6, 7, 8, 9, 10 or more than 10 hooks can be located within a cross-sectional circumferential arc of valve prosthesis 10. One or more hooks 146 can extend radially from the outer circumference of valve prosthesis 10 or from a portion of valve prosthesis 10. Each hook 146 can extend 0.1 mm to 5 mm, from 0.5 mm to 4.5 mm, from 1 mm to 4 mm, from 1.5 mm, to 3.5 mm, from 2 mm to 3 mm in radial distance from the outer circumference of valve prosthesis 10 or from a portion thereof. In some cases, the distance 138 from a distal arch 116 of frame structure 12 and the most distal point on a coil grabber couple to the distal arch can be from 0.1 mm to 5 mm, from 0.5 mm to 4.5 mm, from 1 mm to 4 mm, from 1.5 mm, to 3.5 mm, from 2 mm to 3 mm. FIG. 7 shows valve prosthesis 10A with anchor 15 wrapped around frame structure 12 and resting on hooks 146.

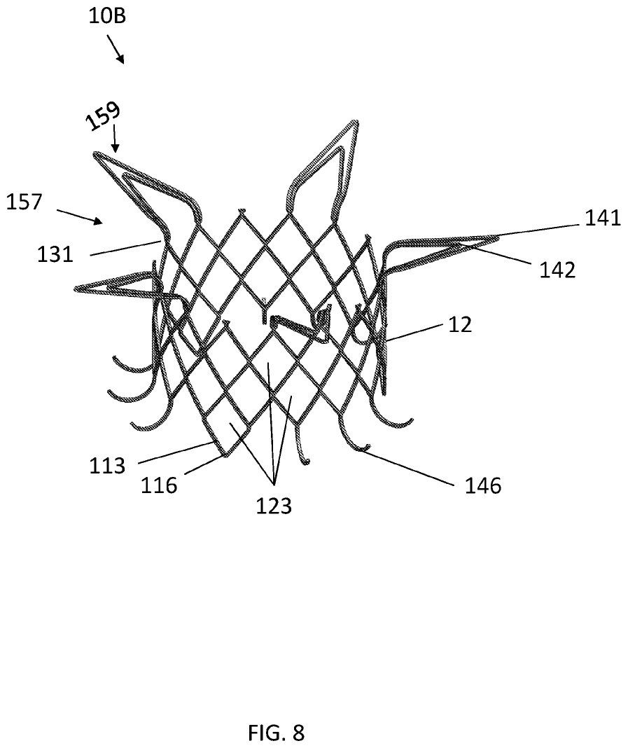

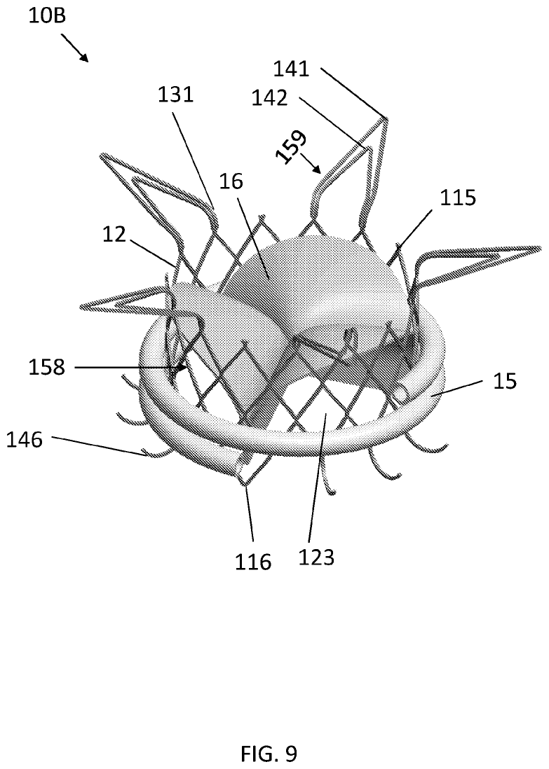

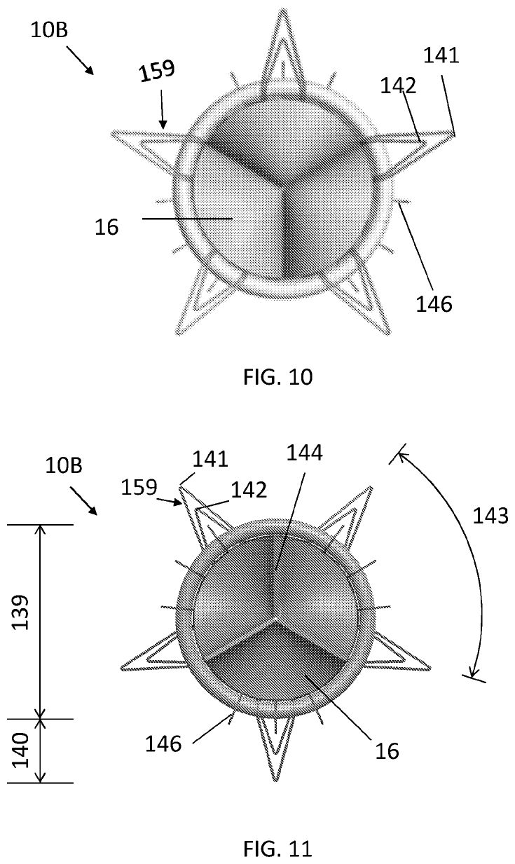

Another exemplary valve prosthesis 10B is shown in FIGS. 8-12. Valve prosthesis 10B is similar to prosthesis 10A except that the atrial flared portion 157 includes five distinct flanges 159.

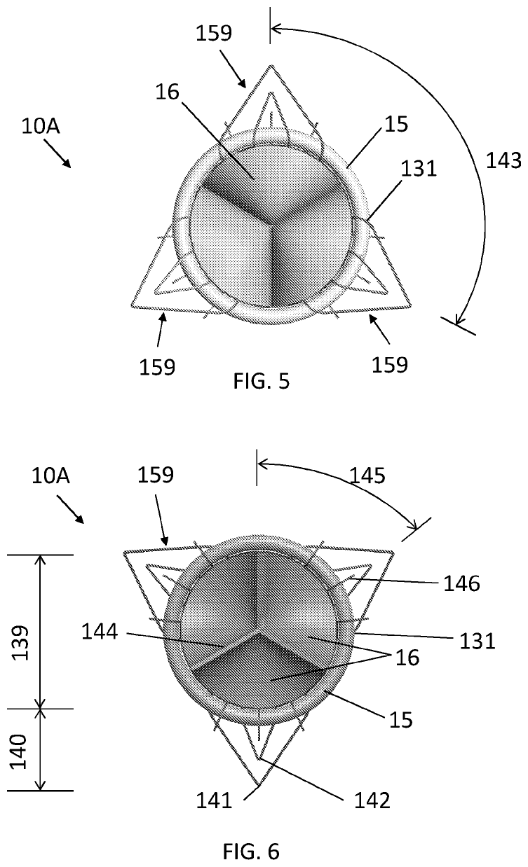

Referring to FIG. 11, the circumferential angle 143 between a first flange 159 and a second flange 159 of the valve prostheses described herein can depend on how many flanges are present on (e.g., coupled to) the valve prosthesis 10A. That is, in general, circumferential angle 143 will be smaller for valve prostheses having more flanges 159. For example, circumferential angle 143 for a valve prosthesis 10B having five flanges 159 (e.g., as shown in FIG. 11) will be smaller than the circumferential angle 143 for a valve prosthesis 10A having three flanges 159 (e.g., as shown in FIG. 5). The circumferential angle 143 between a first flange 159 and a second flange 159 can be from 10 degrees to 180 degrees, from 20 degrees to 150 degrees, from 30 degrees to 120 degrees, from 45 degrees to 90 degrees, or from 60 degrees to 72 degrees.

In some embodiments, the proximal and/or distal arches 115/116 can be flared relative to the central annular portion 158 to form flanges of the frame structure 12.

In some embodiments, valve segment 14 can be disposed entirely or partially at a longitudinal position between the most proximal portion of proximal arches 115 and the most distal portion of distal arches 116 of the central annular portion 158. In other embodiments, all or a portion of valve segment 14 is disposed at a longitudinal position more proximal than any portion of proximal arches 115 of central annular portion 157. In some cases, it is possible for a portion of valve segment 14 to be disposed at a longitudinal position more distal than any portion of distal arches 116.

It will be understood by the person skilled in the art that, although anchor 15 is shown in FIGS. 4-7 and 9-12, anchor 15 is not necessarily coupled directly to prosthetic valve device (10, 10A, or 10B) or a portion thereof. Rather, the atrial flared portion 157 and/or ventricular hooks 146 can be used to capture the anchor 15 (with tissue sandwiched therebetween).

The valve prostheses 10 described herein may comprise a first and second opposite ends, the first end (e.g., the proximal end) oriented nearest the atrium when the valve prosthesis 10 is deployed in the orifice of a native mitral valve and the second end (e.g., the distal end) oriented nearest the ventricle when the valve prosthesis 10 is deployed in the orifice of a native mitral valve. Alternatively, the frame structure 12 may be configured to sit entirely below the native valve when the frame structure 12 is anchored to the native valve. In some cases, a first portion of frame structure 12 can be disposed in a longitudinal location nearer to a first end of the valve prosthesis 10 than the second portion of frame structure 12 (e.g., when the frame structure is in an unexpanded configuration). A first portion and/or second portion of frame structure 12 can have a first longitudinal end and a second longitudinal end. In some cases, a first longitudinal end of frame structure 12 can be oriented nearer to a first end of valve prosthesis 10 than a second longitudinal end of frame structure 12. In some cases, a second longitudinal end of frame structure 12 is oriented nearer to a second end of valve prosthesis 10 than a first longitudinal end of frame structure.

Any of the frame structures 12 described herein can provide structural strength to valve prosthesis device 10 (which can also correspond to 10A, 10B, or any other valve prosthesis described herein). For example, the frame structure 12 can be used to anchor the valve prosthesis 10 in position at a target location of a subject (e.g., in the orifice of a heart valve, such as a mitral valve or tricuspid valve). At least a portion of frame structure 12 can be a rigid (e.g., stiff) or semi-rigid (e.g., resilient or flexible) structure. In various embodiments, a rigid portion of frame structure 12 or portion thereof can be from 0.01 mm to 50 mm in longitudinal length, from 1 mm to 45 mm in longitudinal length, from 10 mm to 40 mm in longitudinal length, from 20 mm to 30 mm in longitudinal length, from 30 mm to 40 mm in longitudinal length, from 25 mm to 35 mm in longitudinal length, from 27.5 mm to 32.5 mm in longitudinal length, from 10 mm to 20 mm in longitudinal length, from 0.01 mm to 10 mm in longitudinal length, from 0.01 mm to 9 mm in longitudinal length, from 0.01 mm to 8 mm in longitudinal length, from 0.01 mm to 7 mm in longitudinal length, from 0.01 mm to 6 mm in longitudinal length, from 0.01 mm to 5 mm in longitudinal length, from 0.01 mm to 4 mm in longitudinal length, from 0.01 mm to 3 mm in longitudinal length, from 0.01 mm to 2 mm in longitudinal length, or from 0.01 mm to 1 mm in longitudinal length. In some cases, the rigid portion of the frame structure 12 is no more than 10 mm in longitudinal length, no more than 9 mm in longitudinal length, no more than 8 mm in longitudinal length, no more than 7 mm in longitudinal length, no more than 6 mm in longitudinal length, no more than 5 mm in longitudinal length, no more than 4 mm in longitudinal length, no more than 3 mm in longitudinal length, no more than 2 mm in longitudinal length, or no more than 1 mm in longitudinal length. The rigid portion of frame structure 12 can comprise one or more struts, one or more arches, one or more commissural posts, one or more leaflet hoops (e.g., hoop structures), one or more flange struts, one or more flange bends, one or more coil grabbers, and/or one or more anchors. In some cases, the second portion 130 of valve prosthesis device 10 is configured to affix to the native valve, e.g., with the aid of an outer anchor, such as anchor 15.

The frame structure 12 of the valve prostheses 10 described herein can comprise a plurality of struts 113 (e.g., arranged into a lattice of expandable cells 123, such as diamond-shaped expandable cells 123). In some cases, the struts 113 are rigid (e.g., stiff).

In some embodiments, as shown in FIG. 1, frame structure 12 can further comprise one or more commissural struts 117. In some cases, commissural strut 117 can serve to join a first portion of frame structure 12 with a second portion of frame structure 12 and/or to attach commissures of the leaflets 16 thereto. In some cases, commissural strut 117 is connected to a portion of the first section 129 of valve prosthesis 10 (e.g., minimal valve support 124), for example, at joint 125. In some cases, the commissural strut 117 can include a gap, slit, or hole (e.g., commissural slit 120, medial hole 118, or distal hole 119) for connection of the leaflet commissures thereto. In some cases, the commissural strut 117 can have a greater transverse cross-sectional area than a strut 113 of frame structure 12. In some cases, the commissural strut 117 can be used to determine the rotational orientation of valve prosthesis 10 around longitudinal axis 152, e.g., during insertion, placement, or anchoring of valve prosthesis 10 at a target region.

In some cases, the one or more commissural struts 117 can be used to align the valve segment 14 with the frame structure 12, for example, with each commissure of the plurality of valve leaflets being aligned with a respective commissural strut 117 as shown in FIG. 1.

In some cases, the struts 113 can be semi-rigid (e.g., resilient or flexible). One or more struts 113 can be oriented and/or connected to one or more other struts 113 to provide structural strength to frame structure 12. For example, one or more strut 113 of frame structure 12 can be oriented parallel to a longitudinal axis to provide structural strength to the frame structure (e.g., in response to compressive force in a longitudinal or substantially longitudinal direction relative to the longitudinal axis 152 of the frame structure). One or more struts 113 can be oriented in a circumferential or substantially circumferential direction relative to frame structure 12 (e.g., residing in a plane that is perpendicular or substantially perpendicular to a longitudinal axis 152). One or more struts 113 of a frame structure 12 can be oriented at an angle relative to a cross-sectional plane that is perpendicular to longitudinal axis 152 of the valve prosthesis.

In some cases, the angle of a strut 113 relative to a cross-sectional plane that is perpendicular to longitudinal axis 152 can depend on the configuration of frame structure 12 and/or valve prosthesis device 10. For example, one or more struts 113 of frame structure 12 may be more perpendicular to a cross-sectional plane (e.g., that is perpendicular to longitudinal axis 152) when the frame structure 12 is in an unexpanded configuration than when the frame structure 12 is in an expanded position. In some cases, the angle between a strut 113 of frame structure 12 and a cross-sectional plane perpendicular to longitudinal axis 152 is from 45 degrees to 55 degrees, from 40 degrees to 60 degrees, from 35 to 65 degrees, from 30 degrees to 70 degrees, from 25 degrees to 75 degrees, from 20 degrees to 80 degrees, from 15 degrees to 85 degrees or from 0 degrees to 90 degrees when frame structure 12 is in an expanded configuration. In some cases, the angle of one or more strut 113 of frame structure 12 relative to a cross-sectional plane perpendicular to longitudinal axis 152 is the same or approximately the same when frame structure 12 is in an expanded configuration versus when frame structure 12 is in an unexpanded configuration.

A first frame element (e.g., strut 113, commissural post 117, or minimal valve support 124) of frame structure 12 can be connected to a second frame element (e.g., strut 113, commissural post 117, or minimal valve support 124) of frame structure 12 (e.g., at a strut joint 114). Frame structure 12 can include a plurality of struts 113 connected at a plurality of strut joints 114 (e.g., forming a lattice structure, which can comprise a portion of frame structure 12). A first frame element can be coupled rigidly to a second frame element. For example, strut joint 114 can comprise a weld or fastener. In some cases, a rigid strut joint 114 does not allow one or more struts 113 connected to the joint 114 to move freely at the joint 114. In some cases, a rigid strut joint 114 can be continuous with one or more struts 113 connected to the joint 114. In some embodiments, the presence of one or more rigid strut joint 114 can increase the resiliency of frame structure 12. In some cases, a first strut 113 can be coupled non-rigidly to a second strut 113 at strut joint 114. For example, strut joint 114 can be a pin joint (e.g., wherein one or more strut 113 connected at the strut joint 114 can rotate freely in a plane around the strut joint 114). In some embodiments, a non-rigid strut joint 114 can improve the ability of frame structure 12 to assume an unexpanded configuration (e.g., inside of a delivery shaft or catheter sheath).

One or more strut 113 of frame structure 12 can comprise a bend or angle. For example, one or more strut 113 of frame structure can for an arch (e.g., an end-most apex of a cell 123), such as distal arch 116 or proximal arch 115 of the central annular portion 158. In some cases, a bend or angle in a strut (such as proximal arch 115 or distal arch 116) can increase the resilience or structural strength of the frame structure 12. In some cases, the orientation of a bend or angle in a strut can influence the directionality of the mechanical properties that it contributes to frame structure 12. FIG. 1 shows a representative example of proximal arches 115 and distal arches 116 that can provide circumferential resilience and or strength to frame structure 12. In some cases, a bend or angle in a first strut 113 can extend a distance 138 past a bend or angle in a second strut 116, longitudinally. In some cases, distal arch 116 can be an attachment point for a commissural strut. In some cases, one or more distal arches 116 can extend a distance 138 past a distal end of a longitudinal strut 113 of the frame structure (and a distance 138 beyond a neighboring distal arch 116), for example, as shown in FIG. 4. In some embodiments, distance 138 is 1%, 2%, 3%, 4%, 5%, 10%, 20%, 30%, from 1% to 5%, from 5% to 20% from 20% to 30%, greater than 30% or less than 1% of the height of the frame body 137.

As noted, the frame structure 12 can comprise one or more expandable cells 123. The frame structure 12 or portion thereof (e.g., a lattice structure of frame structure 12) can comprise 1, 2, 3, 4, 5, 6, 7, 8, 9, 10, from 10 to 20, from 20 to 30, from 30 to 40, from 40 to 50, from 50 to 100, from 100 to 150, from 150 to 200, or more than 200 expandable cells. The lattice structure or portion thereof can have a longitudinal length 137 of from 1 mm to 50 mm, from 1 mm to 45 mm, from 1 mm to 40 mm, from 1 mm to 35 mm, from 1 mm to 30 mm, from 1 mm to 25 mm, from 1 mm to 20 mm, from 1 mm to 10 mm, from 10 mm to 45 mm, from 20 mm to 45 mm, from 20 mm to 30 mm, from 25 mm to 35 mm when valve prosthesis 10 is in an unexpanded configuration. In some cases, a lattice structure or portion thereof can have a longitudinal length of from 1 mm to 45 mm, from 10 mm to 45 mm, from 15 mm to 45 mm, from 15 mm to 35 mm, from 16 mm to 34 mm, from 17 mm to 33 mm, from 18 mm to 32 mm, from 19 mm to 31 mm, from 20 mm to 30 mm, from 25 mm to 35 mm, or from 27.5 mm to 32.5 mm when valve prosthesis 10 is in an expanded configuration. In some cases, an expandable cell 123 of a lattice structure can be defined by the plurality of struts 113 (e.g., wherein the sides of the expandable cells are formed by struts 113). In some cases, an expandable cell 123 can be defined by one or more strut 113 and one or more additional structure, such as a specialized strut (e.g., commissural post 117 or minimal valve support 124). In a representative example, an expandable cell 123 of a frame structure 12 can be in plane with the struts 113 defining the expandable cell 123 (e.g., oriented in a circumferential direction relative to the frame structure). An expandable cell 123 of the frame structure 12 can be of any shape, including square, rectangular, circular, oval, trapezoidal, rhomboid, diamond-shaped, star-shaped, triangular, pentagonal, and hexagonal. As shown in FIG. 1 for example, the expandable cells 123 of the frame structure 12 may be diamond-shaped, with the diamond-shaped expandable cells being circumferentially adjacent one another and the vertices of immediately adjacent expandable cells touching one another.

FIG. 1 shows a frame structure comprising a single row of diamond-shaped expandable cells 123 while FIG. 4 shows two rows of expandable cells 123. However, it is contemplated that frame structure 12 or a portion thereof (e.g., a lattice structure of frame structure 12) may comprise 1, 2, 3, 4, 5, 6, 7, 8, 9, 10, or more than 10 rows of expandable cells 123. In some cases, it can be advantageous to minimize the longitudinal length of a rigid portion of frame structure 12 (e.g., for ease of introduction into a target region of a subject and/or for minimization of total amount of material required to form the frame structure 12). In some cases, longitudinal length of frame structure 12 or portion thereof can be minimized by minimizing the number of rows of expandable cells 123 of frame structure 12 or a portion thereof (e.g., a lattice structure of frame structure 12).

In some cases, expandable cell 123 can be transitioned from a first configuration to a second configuration. For example, expandable cell 123 can be transitioned from an unexpanded configuration to an expanded configuration, e.g., by expanding a balloon or malecot or by allowing one or more structure related to expandable cell 123 (e.g., a strut 113 forming all or a portion of the boundary or edge of expandable cell 123) to expand (e.g., when a self-expandable prosthetic valve device, as described herein, is released from a distal end of a delivery device).

An expandable cell 123 can have a plurality of configurations. For example, expandable cell 123 can have a first configuration (e.g., shape) when valve prosthesis 10 (or a portion thereof) is in an unexpanded configuration and a second configuration (e.g., shape) when valve prosthesis 10 (or portion thereof) is in an expanded configuration. A configuration of expandable cell 123 can have one or more measurable dimensions, e.g., first dimension 121 and/or second dimension 122, as shown in FIG. 1. A dimension (e.g., first dimension 121 or second dimension 122) of expandable cell 123 of frame structure 12 may be different when the frame structure 12 is in an expanded configuration than when the frame structure 12 is in an unexpanded configuration. One or more dimension of expandable cell 123 can be sized to aid in placement and/or anchoring of valve prosthesis 10 at a target region. First dimension 121 or second dimension 122 of expandable cell 123 can be a width, a height, a circumferential arc length, a diameter, a major axis length, a minor axis length, or a radius. In some cases, a dimension of expandable cell 123 (e.g., first dimension 121 or second dimension 122) is from 0.1 mm to 12.0 mm, from 0.1 mm to 5.0 mm, from 0.1 mm to 4.0 mm, from 0.1 mm to 3.5 mm, from 0.1 mm to 3.0 mm, from 0.1 mm to 2.5 mm, from 0.1 mm to 2.0 mm, from 1 mm to 4.0 mm, from 1.0 mm to 3.5 mm, from 1.0 mm to 3.0 mm, from 1.0 mm to 2.0 mm, or from 1.5 mm to 2.5 mm when valve prosthesis 10 is in an unexpanded configuration. In some cases, a dimension of expandable cell 123 (e.g., first dimension 121 or second dimension 122) is from 0.1 mm to 12 mm, from 1.0 mm to 12.0 mm, from 2.0 mm to 12.0 mm, from 3.0 mm to 12.0 mm, from 4.0 mm to 12.0 mm, from 4.5 mm to 11.0 mm, from 5.0 mm to 10.0 mm, from 5.5 mm to 9.0 mm, from 6.0 mm to 8.0 mm, from 6.5 mm to 7.0 mm, from 5.0 mm to 12.0 mm, or from 6.0 mm to 12.0 mm, from 7.0 mm to 12.0 mm, from 8.0 mm to 12.0 mm, from 9.0 mm to 12.0 mm, from 10.0 mm to 12.0 mm, or from 11.0 mm to 12.0 mm when valve prosthesis 10 is in an expanded configuration.

Frame structure 12 can have a round (e.g., circular) cross-section or polygonal cross-section (e.g., relative to longitudinal axis 152). The cross-sectional shape of frame structure 12 at a longitudinal position along longitudinal axis 152 of frame structure 12 can be influenced by the number and/or spacing of struts 113 along the perimeter of frame structure 12 at the longitudinal position. At a given point along longitudinal axis 152, struts 113 can be arranged in a single layer around the circumference of frame structure 12 or portion thereof. For example, a cross-section of frame structure 12 can comprise just one strut 113 at a given angle in a cross-sectional plane perpendicular to longitudinal axis 152 at a given point along longitudinal axis 152. A frame structure 12 comprising only one layer of struts in a cross-sectional plane perpendicular to longitudinal axis 152 can often reduce the amount of material required to form the frame structure 12 while retaining sufficient strength (e.g., in stenting open a native valve, native vessel, or other native tissue). At one or more points along the longitudinal axis 152 of frame structure 12, frame structure 12 can comprise more than one strut 113 per arc angle in a cross-sectional plane perpendicular to the longitudinal axis 152 as the one or more points along longitudinal axis 152. For example, a frame structure can comprise 2, 3, 4, 5, 6, 7, 8, 9, 10, or more than 10 layers of struts at an arc angle in a cross-sectional plane perpendicular to longitudinal axis 152. In some cases, a frame structure 12 comprising more layers of struts (e.g., a double layer or triple layer of struts) at a point along longitudinal axis 152 may have improved strength compared to a frame structure comprising only one layer of struts at a point along longitudinal axis 152.

It is further contemplated that a frame structure 12 or portion thereof can comprise a first expandable cell 123 having a first shape and/or set of dimensions and a second expandable cell 123 having a second shape and/or set of dimensions. In some cases, the shape of the first expandable cell 123 is the same as the shape of the second expandable cell 123. In some cases, the shape of the first expandable cell 123 is different than the shape of the second expandable cell 123. A first expandable cell 123 of frame structure 12 can have one or more dimensions equal to a second expandable cell 123 of frame structure 12. In some case, one or more dimensions of a first expandable cell 123 of frame structure 12 is different than one or more dimensions of a second expandable cell 123 of frame structure 12 (e.g., one or more corresponding dimensions of second expandable cell 123).

In some cases, the cross-sectional shape of strut 113 can impact the structural strength that strut 113 provides to frame structure 12. Strut 113 can have one of numerous cross-sectional shapes at a point along strut 113, including square, rectangular, circular, oval, trapezoidal, rhomboid, diamond-shaped, star-shaped, triangular, pentagonal, and hexagonal. The cross-sectional shape of strut may have 3, 4, 5, 6, 7, 8, 9, 10, or more than 10 sides. In some cases, the cross-sectional shape of strut 113 can be different at a first point along strut 113 than at the cross-sectional shape of strut 113 at a second point along strut 113. For example, strut 113 can have a circular cross-section at a first point along strut 113 and a rectangular cross-section at a second point along strut 113. In some cases, the cross-sectional shape of strut 113 can improve the strength and/or resilience of strut 113 with respect to a certain type of force or stress (e.g., a compressive force or stress, a tensional force or stress, a torsional force or stress, a bending force or stress, or a shearing force or stress) or a directionality of a force or stress (e.g., a force or stress applied perpendicularly, in parallel to, or obliquely to a surface of strut 113) acting upon strut 113. In some cases the cross-sectional shape of strut 113 (or change in cross-sectional shape along strut 113) can aid in reducing the amount of material needed to form or construct strut 113 and/or frame structure 12. In some cases, strut 113 may be hollow or may comprise voids, such as slots, holes, and other gaps.

The cross-sectional shape of strut 113 can refer to one or more cross-sectional dimensions of strut 113. The cross-sectional shape of strut 113, which can refer to the thickness of strut 113, can impact the structural strength that strut 113 provides to frame structure 12. In some cases, a cross-sectional dimension of strut 113 refers to the cross-sectional length or width of strut 113 at a point along strut 113. In some cases, the cross-sectional shape of strut 113 refers to a cross-sectional area of strut 113 at a point along strut 113. One or more cross-sectional dimensions of strut 113 can vary along the length of strut 113. For example, a portion of a strut 113 can have a first cross-sectional area at a first point along strut 113 and a second cross-sectional area at a second point along strut 113. In some cases, the ratio of first cross-sectional area of strut 113 to second cross-sectional area of strut 113 can be from 1:1.1 to 1:3, from 1:1.3 to 1:1.5, from 1:1.5 to 1:2, from 1:2 to 1:3, or from 1:3 to 1:5.

The valve prostheses 10 described herein may include one or more valve segments 14 disposed therein to replace the native valve leaflets. For example, the valve segment 14 can include a plurality of leaflets 16, e.g., that form a biocompatible one-way valve. Flow in one direction may cause the leaflets 16 to deflect open and flow in the opposite direction may cause the leaflets 16 to close.

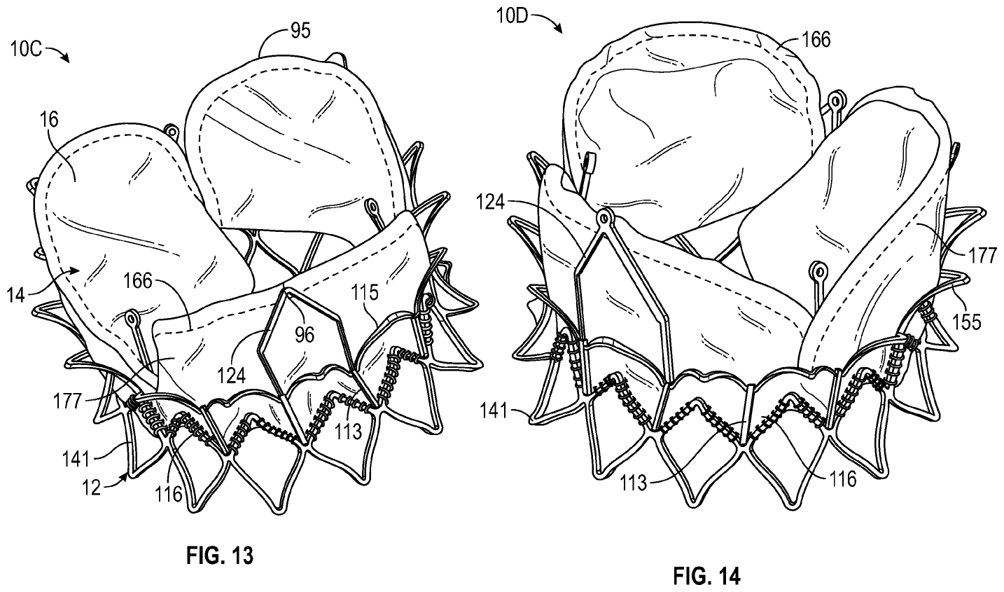

Any of the valve segments 14 described herein may be formed of multi-layered materials for preferential function. Referring to FIG. 13, for example, the valve prosthesis 10C may include a valve segment 14 having a seal 177 (also called an outer leaflet, outer layer, or skirt) positioned radially between leaflets 16 (also called inner leaflets or the inner layer) and the frame structure 12. The seal 177 can be a single piece wrapped around the leaflets 16 or can be individual pieces shaped to match the leaflets 16. In some cases, the seal 177 and/or leaflets 16 can be formed from or coated with a material to confer an advantage upon the valve segment 14. For example, a layer or surface of a valve segment 14 can be formed from or coated with a biocompatible material. In some cases, a layer or surface of a valve segment 14 can be formed from or coated with an anti-thrombotic material. In some cases, a valve segment 14 (or portion thereof, such as a leaflet 16 of the valve segment) comprises a synthetic material. In some cases, a valve segment 14 (or portion thereof, such as a leaflet) comprises from a biological tissue. In many cases, a valve segment 14 (or portion thereof, such as a leaflet) comprises pericardial tissue. In some embodiments, a valve segment 14 (or portion thereof, such as a leaflet 16 of the valve segment 14) comprises a decellularized biological tissue. For example, a valve segment 14 (or portion thereof, such as a leaflet 16 of the valve segment) can include decellularized pericardium.

The valve segment 14 may be attached to a frame structure 12, which can in turn be attached to the anchor 15. The frame structure 12 may be connected to the anchor 15 before or after the frame structure 12 has been deployed adjacent a native valve. The frame structure 12 may be attached to the valve segment 12, for example, via attachment of the frame structure 12 to the seal 177, which can in turn be attached to the leaflets 16.

In some embodiments, two or more portions of a valve segment 15 (e.g., two or more leaflets 16, and/or seal 177) can comprise a single piece of material (e.g., a single piece of biological or synthetic tissue formed into the shape of a functional valve). In some cases, two or more portions of a valve segment (e.g., two or more of a first and second leaflet 16, and/or the seal 177) can be joined together. In some embodiments, two or more portions of a valve segment (e.g., two or more of a first and second leaflet 16, and/or the seal 177) can be joined together by suturing the two or more portions together (e.g., at sutured coupling 166 shown in FIG. 13). In some cases, 1, 2, 3, 4, 5, or more than 5 leaflets 16 can be coupled to a single seal 177.

In many cases, leaflet coupling 166 is disposed at an inflow end of valve prosthesis 10 (i.e., closest to the source of flow through the device, e.g., caused by a contracting heart chamber) when deployed. In some cases, coupling two or more portions of a valve segment 14 at the inflow end of valve prosthesis 10 (or portion thereof) allows the valve segment 14 to fold or collapse (e.g., radially away from a longitudinal axis of valve prosthesis device 10) during contraction of a heart chamber upstream of the deployed device (i.e., during diastole). Further, in some cases, coupling two or more portions of a valve segment 14 at the inflow end of valve prosthesis 10 causes the valve segment 14 to expand (e.g., radially toward a longitudinal axis of valve prosthesis device 10) during refilling of a heart chamber upstream of the deployed device (i.e., during systole). This expansion of the valve segment 14 can, for example, result in billowing or parachuting of the valve segment 14 (e.g., between the seal 177 and the leaflets 16) to block the flow of blood therethrough.

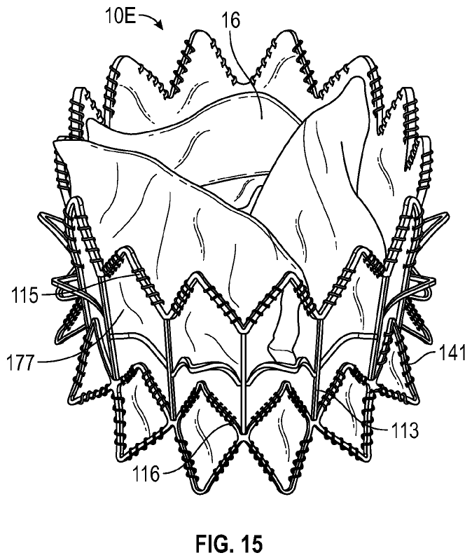

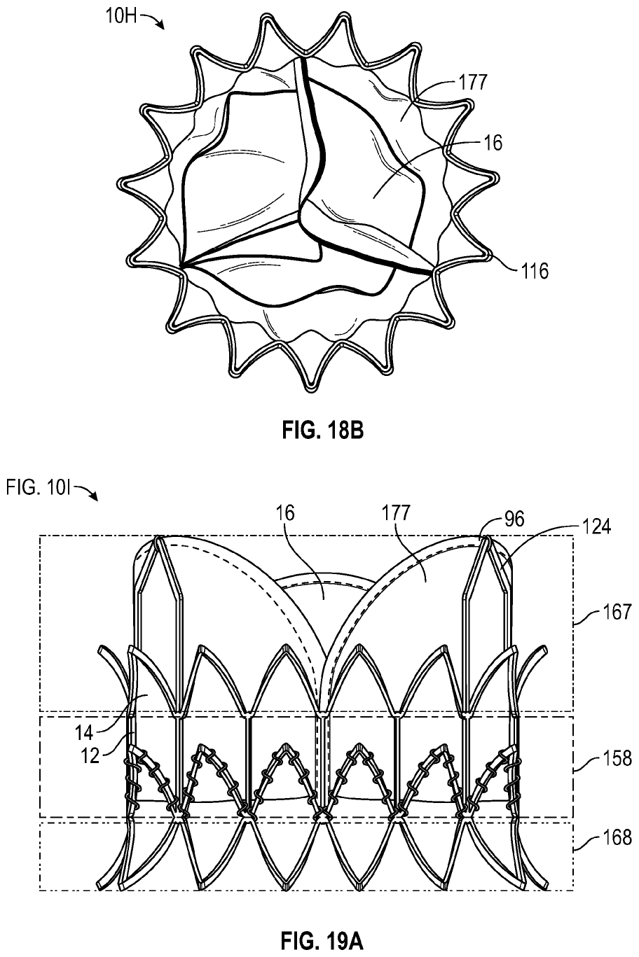

As shown in to FIG. 13, the valve segment 14 can be attached to one or more struts 113 of the frame structure 12. In some embodiments, a portion of a valve segment 14 (e.g., leaflets 16 or seal 177) can be sutured to the central annular portion 158 of frame structure 12 and not to the inflow portion of frame structure 12 or the outflow portion of frame structure 12 (e.g., can be unattached to the distal arches 116 and the proximal arches 115 as shown in FIG. 13). In some embodiments, a portion of a valve segment 14 (e.g., leaflets 16 or seal 177) can be sutured to one or more outflow portion of frame structure 12 and not to the inflow portion of frame structure 12 (e.g., can be sutured to one or more distal arches 116 but not one or more proximal arches 115 as shown in FIG. 18A). In some embodiments, a portion of a valve segment 14 (e.g., leaflets 16 or seal 177) can be sutured to one or more outflow portion of frame structure 12 and to the inflow portion of frame structure 12 (e.g., can be sutured to one or more distal arches 116 and also to one or more proximal arches 115 as shown in valve prosthesis 10E of FIG. 15). In some embodiments, an inflow end of the valve segment 14 can be substantially unsupported by the frame 12 while the outflow end of the valve segment 14 can be fully supported by and within the valve segment 14 (as shown in FIG. 13). The valve segment 14 (or portion thereof, such as the seal 177) can be coupled to the frame 12 continuously around the inner circumference of the frame 12 (e.g., at a distal or outflow end of valve prosthesis device 10).

In some cases, the amount of attachment of a valve segment 14 (e.g., a valve leaflet 16) to the frame structure 12 can be minimized, which can advantageously enhance ease of delivery and reduce the required length of the frame, thereby reducing the chance of thrombosis and reducing the chance of blocking the outflow from the ventricle to the aorta. Minimizing the frame structure 12 can also improve the speed and cost of fabrication of the valve prosthesis device 10.

In some embodiments, a leaflet 16 that is attached to a first portion of frame structure 12 (e.g., one or more struts 113) at a distal end of frame structure 12 can be unattached at a proximal end of the frame structure 12 (e.g., a strut or portion thereof at a proximal end of frame structure 12). In some cases, valve prosthesis devices 10 in which a valve segment 14 is attached at a proximal end of frame structure 12 and is unattached at a proximal end of frame structure 12 (and/or at a proximal end of valve segment 14) may require less metal and/or fewer struts than a valve prosthesis 10 in which a valve segment 14 is attached at both a proximal end and a distal end of the frame structure 12 of the valve prosthesis device 10. In some cases, minimizing the amount of metal used in the structure of valve prosthesis 10 (e.g., by reducing the number and/or length of struts in valve prosthesis device 10) can reduce the risk of thrombus formation and can improve the ease with which the device is deployed at a target location.

Further, the valve segment 14 can be configured to be substantially unsupported at the inflow edge 95 of the valve segment 14. For example, as shown in FIG. 13, the entire inflow edge 95 of valve segment can be unsupported with the exception of minimal valve supports 124 positioned at the nadir 96 of each leaflet 16. The valve supports 124 can have a pointed proximal tip and can extend, for example, from two neighboring struts 113 of the frame structure 12. The minimal valve supports 124 can help prevent the valve segment 14 (e.g., the seal) from collapsing radially inwards in the outflow direction (i.e., towards the ventricle) when implanted in the heart. FIG. 14 shows a valve prosthesis 10D that is similar to valve prosthesis 10C of FIG. 13 except that the valve support 124 of FIG. 14 includes an aperture 97 for suturing the leaflet 16 to the valve support 124.

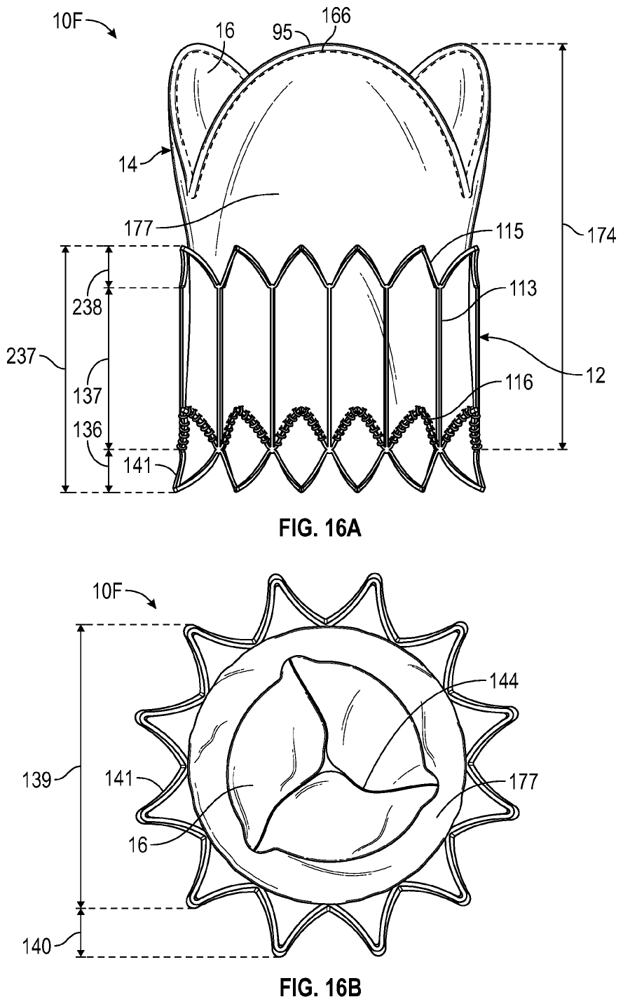

FIGS. 16A-16B show a valve prosthesis 10F wherein the inflow edge 95 of valve segment 14 is completely unsupported (i.e., does not include any valve supports thereto).

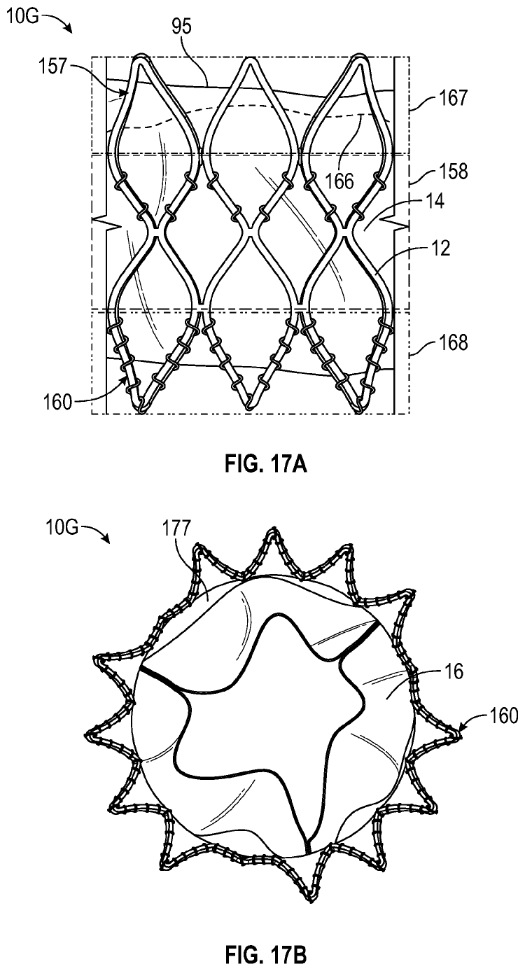

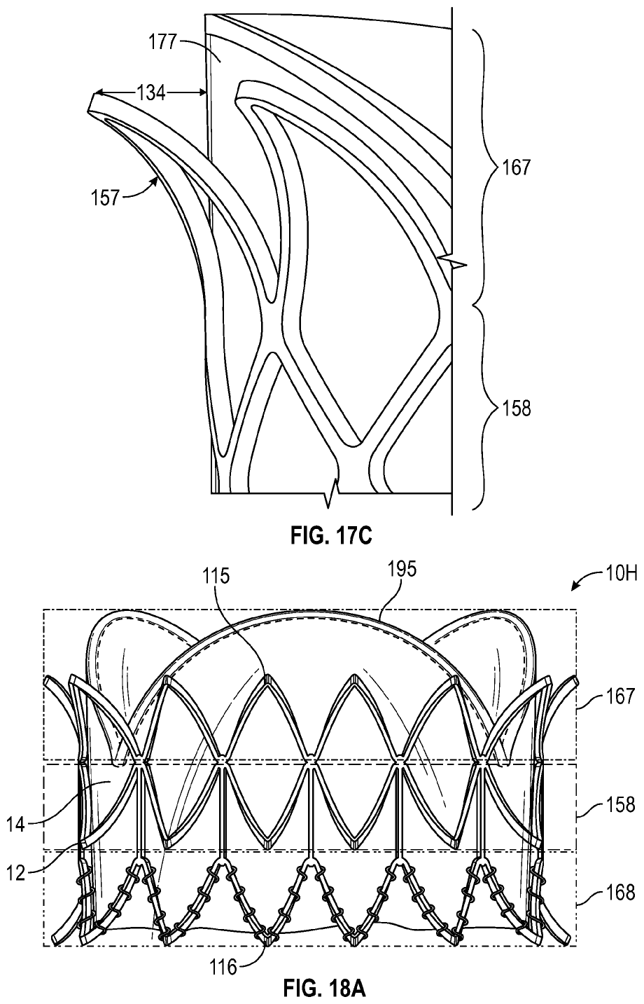

FIGS. 17A-17C show another valve prosthesis 10G wherein the inflow edge 95 of valve segment 14 is completely unsupported (i.e., does not include any valve supports thereto). Indeed, as shown in FIGS. 17A-17C, the prosthesis 10G can include an inflow portion 167, a central annular portion 158, and an outflow portion 168. The valve segment 14 can be fully circumferentially supported by the frame structure 12 within the central annular section 158. However, the valve segment 14 can be unsupported by and/or unconnected from the frame structure 12 in the inflow section 167. Further, the frame structure 12 can flare radially outwards within the inflow section 167. The flared portion 157 of the frame structure 12 can include a plurality of discrete flanges (i.e., formed from flared proximal arches 115) and can, for example, serve to help engage with an external anchor. Moreover, due to the flared portion 157, the valve segment 14 can be radially spaced away from the frame structure 12 within the inflow section 167 by a distance 134 (see FIG. 17C). In some embodiments, the distance 134 can be 1-10 mm, such as 2-8 mm, such as 3-5 mm. Finally, the frame structure 12 can also flare radially outwards within the outflow section 168. The flared portion 160 of the frame structure 12 can also serve to help engage with an external anchor 15. For example, the external anchor 15 can sit between the flared portions 157, 160 upon implantation.

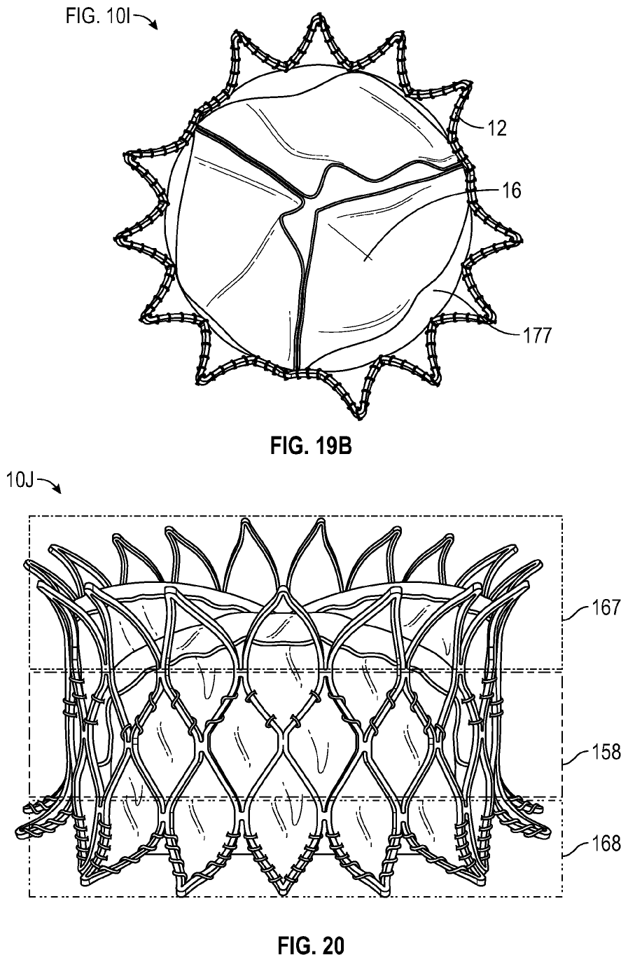

FIG. 20 shows another valve prosthesis 10J that is similar to valve prosthesis 10G of FIGS. 17A-17B except that the ratio of width of the cells (i.e., in the circumferential direction) to height of the cells can be greater in valve prosthesis 10J than valve prosthesis 10G. The dimensions of the cells can be modified to provide the desired stiffness and stability.

FIGS. 18A-18B show another valve prosthesis 10H that is similar to valve prosthesis 10G of FIGS. 17A-17C except that substantially all of the inflow edge 95 extends proximally beyond the proximal arches 15 of the frame structure 12. When the leaflets are closed (as shown in FIG. 18B), the fluid pressure can act to fill the space created by the leaflets 16 and the seal 177, thereby preventing inward motion or collapse of the valve segment 14.

FIGS. 19A-19B show a valve prosthesis 10I that is similar to valve prosthesis 10H of FIGS. 18A-18B except that it includes a minimal valve support 124 at the nadir 96 of each leaflet 16. The minimal valve supports 124 are similar to the valve supports 124 of FIG. 13. Additionally, the valve segment 14 of valve prosthesis 10I ends before the start of the outflow section 167 (i.e., ends within the central annular section 158). Not having the valve segment 14 attached at the outflow section 167 may advantageously reduce tension on the frame structure 14 where the frame structure 14 engages the external anchor 15 (i.e., within the outflow section 167).

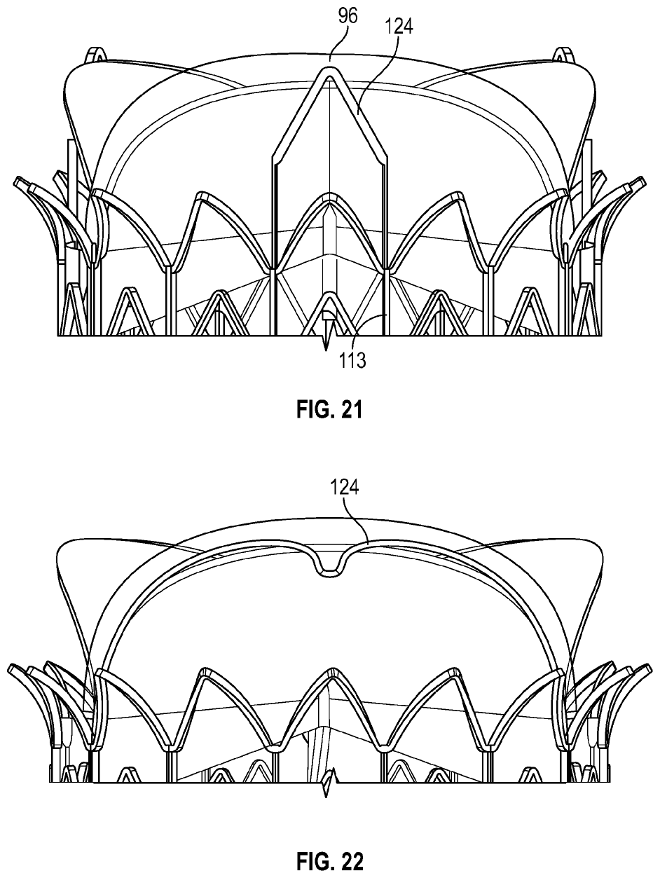

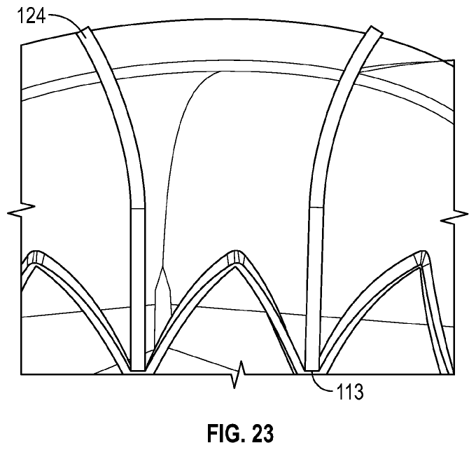

Various embodiments of minimal valve supports 124 are shown in FIGS. 21-22. For example, as shown in FIG. 21, the valve support 124 can extend from one or more longitudinal struts 113 and attach to the valve segment 14 at the nadir 96. As shown in FIG. 22, the minimal valve support 124 can be a hoop support that extends only along the inflow edge 95, but otherwise leaves the valve segment 14 unsupported within the inflow section. As shown in FIG. 23, the valve supports 124 can be wire forms that extend longitudinally from one or more longitudinal struts 113. The minimal valve supports 124 can advantageously help prevent partial prolapse of the valve segment 14 while still keeping the majority of the valve segment 14 in the inflow section unsupported.

In some embodiments, the minimal valve supports 124 (e.g., those shown in FIGS. 21-22) can be positioned between the leaflets 16 and the seal 177. Having the minimal valve supports 124 protected within the valve segment 14 between the leaflets 16 and the seal 177 may advantageously making loading and/or releasing from the delivery system easier (e.g., by reducing friction and/or catching). Further, in some embodiments, the minimal valve supports 124 can be hinged at the connection to the frame 124 to assist in loading and/or releasing from the delivery system. In some embodiments, the minimal valve supports 124 that are positioned between the leaflets 16 and the seal 177 can be formed of a coil to help prevent kinking.

In some embodiments, the inflow edge 95 can be entirely unsupported except at commissures of the leaflets 16. In some embodiments, the inflow edge 95 can be unsupported except at commissures of the leaflets 16 and the valve supports 124.

Referring to FIGS. 16A-16B, in some cases, the size of a valve prosthesis 10F (which can correspond to any of the valve prostheses 10 described herein), e.g., the magnitude of a frame height 137 of a valve prosthesis 10F in an expanded configuration) can be measured relative to one or more structures of the valve prosthesis 10F (e.g., a valve segment height of the valve prosthesis device in an expanded configuration, a leaflet height 174 when the device is expanded, and/or a diameter 139 of an expanded frame body) and/or relative to one or more biological structures (e.g., the mean diameter of a heart valve in which the device is deployed).

In some cases, the height 137 of a frame of the valve prosthesis 10F can be measured relative to the height 174 of a valve segment 14 of the valve prosthesis device 10F (e.g., valve segment height-to-frame height ratio, or VSTF ratio, e.g., a ratio of height 137 to height 174). In some cases, the height 174 of a valve segment 14 (or portion thereof, such as a valve leaflet) of an expanded valve prosthesis 10F is greater than the height of the frame of the valve prosthesis device (e.g., a VSTF ratio greater than 1). In some cases, the height 174 of a valve segment 14 (or portion thereof) of a valve prosthesis 10F in an expanded configuration is 1% larger than to 1000% larger than the frame height of the valve prosthesis device. In some cases, the height 174 of a valve segment (or portion thereof, such as a valve leaflet) of a valve prosthesis 10F in an expanded configuration is 1% to 5%, 5% to 10%, 5% to 15%, 5% to 20%, 5% to 25%, 5% to 30%, 5% to 35%, 5% to 40%, 5% to 45%, 5% to 50%, 5% to 60%, 5% to 70%, 10% to 15%, 10% to 20%, 10% to 25%, 10% to 30%, 10% to 35%, 10% to 40%, 10% to 45%, 10% to 50%, 10% to 60%, 10% to 70%, 15% to 20%, 15% to 25%, 15% to 30%, 15% to 35%, 15% to 40%, 15% to 45%, 15% to 50%, 15% to 60%, 15% to 70%, 20% to 25%, 20% to 30%, 20% to 35%, 20% to 40%, 20% to 45%, 20% to 50%, 20% to 60%, 20% to 70%, 25% to 30%, 25% to 35%, 25% to 40%, 25% to 45%, 25% to 50%, 25% to 60%, 25% to 70%, 30% to 35%, 30% to 40%, 30% to 45%, 30% to 50%, 30% to 60%, 30% to 70%, 35% to 40%, 35% to 45%, 35% to 50%, 35% to 60%, 35% to 70%, 40% to 45%, 40% to 50%, 40% to 60%, 40% to 70%, 45% to 50%, 45% to 60%, 45% to 70%, 50% to 60%, 50% to 70%, 60% to 70%, 70% to 80%, 80% to 90%, 90% to 100%, 100% to 110%, 110% to 120%, 120% to 130%, 130% to 140%, 140% to 150%, 150% to 160%, 160% to 170%, 170% to 180%, 180% to 190%, 190% to 200%, 200% to 250%, 250% to 300%, 300% to 400%, 400% to 500%, 500% to 600%, 600% to 700%, 700% to 800%, 800% to 900%, 900% to 1000%, or more than 1000% of the height of the frame structure 12 of the valve prosthesis 10. In some cases, a valve prosthesis 10F in an expanded configuration has a valve segment height-to-frame height ratio (e.g., VSTF ratio) of 1.01 to 1.1, 1.1 to 1.2, 1.2 to 1.3, 1.3 to 1.4, 1.4 to 1.5, 1.5 to 1.6, 1.6 to 1.7, 1.7 to 1.8, 1.8 to 1.9, 1.9 to 2.0, 2.0 to 2.5, 2.5 to 3.0, 3.0 to 4.0, 4.0 to 5.0, 5.0 to 6.0, 6.0 to 7.0, 7.0 to 8.0, 8.0 to 9.0, 9.0 to 10.0, or more than 10.0. In some cases, the height 174 of a valve segment (or portion thereof) of a valve prosthesis 10F in an expanded configuration is 5%, 10%, 15%, 20%, 25%, 30%, 35%, 40%, 45%, 50%, 60%, 70%, 80%, 90%, 100%, 110%, 120%, 130%, 140%, 150%, 160%, 170%, 180%, 190%, 200%, 250%, 300%, 400%, 500%, 600%, 700%, 800%, 900%, or 1000% larger than the height of the frame of the valve prosthesis device. In some cases, the height 174 of a valve segment 14 (or portion thereof, such as a valve leaflet 16) of a valve prosthesis 10F in an expanded configuration is at least 5%, 10%, 15%, 20%, 25%, 30%, 35%, 40%, 45%, 50%, 60%, 70%, 80%, 90%, 100%, 110%, 120%, 130%, 140%, 150%, 160%, 170%, 180%, 190%, 200%, 250%, 300%, 400%, 500%, 600%, 700%, 800%, 900%, or 1000% larger than the height of the frame of the valve prosthesis 10. In some cases, the height 174 of a valve segment 14 (or portion thereof, such as a valve leaflet 16) of a valve prosthesis 10 in an expanded configuration is at most 5%, 10%, 15%, 20%, 25%, 30%, 35%, 40%, 45%, 50%, 60%, 70%, 80%, 90%, 100%, 110%, 120%, 130%, 140%, 150%, 160%, 170%, 180%, 190%, 200%, 250%, 300%, 400%, 500%, 600%, 700%, 800%, 900%, or 1000% larger than the height of the frame structure 12 of the valve prosthesis 10F.

In some cases, the height of a valve segment 14 of an expanded valve prosthesis 10F is equal to the height of the frame structure 12 of the valve prosthesis 10F (e.g., a VSTF ratio equal to 1).

In some cases, the height of a valve segment 14 of an expanded valve prosthesis 10F is less than the height of the frame structure 12 of the valve prosthesis 10F (e.g., a VSTF ratio less than 1). In some cases, the height of a valve segment 14 (or portion thereof) of a valve prosthesis 10F in an expanded configuration is 1% larger than to 1000% larger than the frame height of the valve prosthesis 10F. In some cases, the height of a valve segment 14 (or portion thereof, such as a valve leaflet 16) of a valve prosthesis 10F in an expanded configuration is 1% to 5%, 5% to 10%, 5% to 15%, 5% to 20%, 5% to 25%, 5% to 30%, 5% to 35%, 5% to 40%, 5% to 45%, 5% to 50%, 5% to 60%, 5% to 70%, 10% to 15%, 10% to 20%, 10% to 25%, 10% to 30%, 10% to 35%, 10% to 40%, 10% to 45%, 10% to 50%, 10% to 60%, 10% to 70%, 15% to 20%, 15% to 25%, 15% to 30%, 15% to 35%, 15% to 40%, 15% to 45%, 15% to 50%, 15% to 60%, 15% to 70%, 20% to 25%, 20% to 30%, 20% to 35%, 20% to 40%, 20% to 45%, 20% to 50%, 20% to 60%, 20% to 70%, 25% to 30%, 25% to 35%, 25% to 40%, 25% to 45%, 25% to 50%, 25% to 60%, 25% to 70%, 30% to 35%, 30% to 40%, 30% to 45%, 30% to 50%, 30% to 60%, 30% to 70%, 35% to 40%, 35% to 45%, 35% to 50%, 35% to 60%, 35% to 70%, 40% to 45%, 40% to 50%, 40% to 60%, 40% to 70%, 45% to 50%, 45% to 60%, 45% to 70%, 50% to 60%, 50% to 70%, 60% to 70%, 70% to 80%, 80% to 90%, 90% to 100%, 100% to 110%, 110% to 120%, 120% to 130%, 130% to 140%, 140% to 150%, 150% to 160%, 160% to 170%, 170% to 180%, 180% to 190%, 190% to 200%, 200% to 250%, 250% to 300%, 300% to 400%, 400% to 500%, 500% to 600%, 600% to 700%, 700% to 800%, 800% to 900%, 900% to 1000%, or over 1000% less than the height of the frame structure 12 of the valve prosthesis 10F. In some cases, a valve prosthesis 10F in an expanded configuration has a valve segment height-to-frame height ratio (e.g., VSTF ratio) of 0.99 to 0.90, 0.90 to 0.80, 0.80 to 0.70, 0.70 to 0.60, 0.60 to 0.50, 0.50 to 0.40, 0.40 to 0.30, 0.30 to 0.25, 0.25 to 0.20, 0.20 to 0.15, 0.15 to 0.10, 0.10 to 0.01, less than 0.01, or between 1.00 and 0.99. In some cases, the height of a valve segment (or portion thereof) of a valve prosthesis 10F in an expanded configuration is 5%, 10%, 15%, 20%, 25%, 30%, 35%, 40%, 45%, 50%, 60%, 70%, 80%, 90%, 100%, 110%, 120%, 130%, 140%, 150%, 160%, 170%, 180%, 190%, 200%, 250%, 300%, 400%, 500%, 600%, 700%, 800%, 900%, or 1000% less than the height of the frame structure 12 of the valve prosthesis 10F. In some cases, the height of a valve segment 14 (or portion thereof, such as a valve leaflet 16) of a valve prosthesis 10F in an expanded configuration is at least 5%, 10%, 15%, 20%, 25%, 30%, 35%, 40%, 45%, 50%, 60%, 70%, 80%, 90%, 100%, 110%, 120%, 130%, 140%, 150%, 160%, 170%, 180%, 190%, 200%, 250%, 300%, 400%, 500%, 600%, 700%, 800%, 900%, or 1000% smaller than the height of the frame structure 12 of the valve prosthesis 10F. In some cases, the height of a valve segment 14 (or portion thereof, such as a valve leaflet 16) of a valve prosthesis 10F in an expanded configuration is at most 5%, 10%, 15%, 20%, 25%, 30%, 35%, 40%, 45%, 50%, 60%, 70%, 80%, 90%, 100%, 110%, 120%, 130%, 140%, 150%, 160%, 170%, 180%, 190%, 200%, 250%, 300%, 400%, 500%, 600%, 700%, 800%, 900%, or 1000% smaller than the height of the frame of the valve prosthesis 10F.

In various embodiments, it can be important to select an appropriate diameter of a valve prosthesis 10F (or portion thereof, such as a diameter of a frame structure 12 of a valve prosthesis 10F) and/or height of a valve prosthesis 10F (or portion thereof, such as a diameter of a frame structure 12 of a valve prosthesis 10F) in order to minimize deleterious effects of an improper fit in the heart region in which the valve prosthesis 10F is to be employed. In some cases, selection of an appropriate diameter of a valve prosthesis 10F is based on the location in which the valve prosthesis 10F is to be deployed (e.g., which native valve is to be replaced) and/or on a present or previous physiological condition of the patient in whom the valve prosthesis 10F is to be deployed (e.g., the presence or history of heart disease, such as congestive heart failure).

In some cases, the height of a frame structure 12 of a valve prosthesis 10F is measured relative to the diameter of a heart valve in which the valve prosthesis 10F is deployed (e.g., a mitral valve, a tricuspid valve, a pulmonary valve, or an aortic valve). Using one metric to determine diameter of heart valves in patients with or without congestive heart failure (CHD), Westaby et al. (Am J Cardiol 1984; 53:552-556, which is incorporated by reference herein in its entirety and for all purposes) show that a mitral valve in a male patient without CHD can have a diameter of 32.3 mm (standard deviation ±3.3 mm), a mitral valve in a male patient with CHD can have a diameter of 33.3 mm (standard deviation ±4.0 mm), a mitral valve in a female patient without CHD can have a diameter of 29.0 mm (standard deviation ±2.7 mm), and a mitral valve in a female patient with CHD can have a diameter of 30.9 mm (standard deviation ±3.1 mm). Westaby et al. also show that a tricuspid valve in a male patient without CHD can have a diameter of 36.4 mm (standard deviation ±4.4 mm), a tricuspid valve in a male patient with CHD can have a diameter of 39.7 mm (standard deviation ±4.6 mm), a tricuspid valve in a female patient without CHD can have a diameter of 33.2 mm (standard deviation ±3.3 mm), and a tricuspid valve in a female patient with CHD can have a diameter of 36.4 mm (standard deviation ±3.6 mm). Westaby et al. further shows that an aortic valve in a male patient can have a diameter of 23.8 mm (standard deviation ±3.3 mm) and an aortic valve in a female patient can have a diameter of 21.60 mm (standard deviation ±2.8 mm). Westaby et al. shows that a pulmonary valve in a male patient can have a diameter of 24.70 mm (standard deviation ±3.1 mm) and a pulmonary valve in a female patient can have a diameter of 23.3 mm (standard deviation ±2.7 mm).

In some cases, the frame height 137 of an expanded valve prosthesis 10F is greater than the diameter of a heart valve in which the valve prosthesis 10F is deployed. In some cases, the height 137 of the frame structure 12 of a valve prosthesis 10F in an expanded configuration is 1% larger than to 1000% larger than the diameter of the heart valve in which the valve prosthesis 10F is deployed. In some cases, the height of a frame structure 12 of a valve prosthesis 10F in an expanded configuration is 1% to 5%, 5% to 10%, 5% to 15%, 5% to 20%, 5% to 25%, 5% to 30%, 5% to 35%, 5% to 40%, 5% to 45%, 5% to 50%, 5% to 60%, 5% to 70%, 10% to 15%, 10% to 20%, 10% to 25%, 10% to 30%, 10% to 35%, 10% to 40%, 10% to 45%, 10% to 50%, 10% to 60%, 10% to 70%, 15% to 20%, 15% to 25%, 15% to 30%, 15% to 35%, 15% to 40%, 15% to 45%, 15% to 50%, 15% to 60%, 15% to 70%, 20% to 25%, 20% to 30%, 20% to 35%, 20% to 40%, 20% to 45%, 20% to 50%, 20% to 60%, 20% to 70%, 25% to 30%, 25% to 35%, 25% to 40%, 25% to 45%, 25% to 50%, 25% to 60%, 25% to 70%, 30% to 35%, 30% to 40%, 30% to 45%, 30% to 50%, 30% to 60%, 30% to 70%, 35% to 40%, 35% to 45%, 35% to 50%, 35% to 60%, 35% to 70%, 40% to 45%, 40% to 50%, 40% to 60%, 40% to 70%, 45% to 50%, 45% to 60%, 45% to 70%, 50% to 60%, 50% to 70%, 60% to 70%, 70% to 80%, 80% to 90%, 90% to 100%, 100% to 110%, 110% to 120%, 120% to 130%, 130% to 140%, 140% to 150%, 150% to 160%, 160% to 170%, 170% to 180%, 180% to 190%, 190% to 200%, 200% to 250%, 250% to 300%, 300% to 400%, 400% to 500%, 500% to 600%, 600% to 700%, 700% to 800%, 800% to 900%, 900% to 1000%, or more than 1000% of the diameter of a heart valve in which the device is deployed. In some cases, a valve prosthesis 10F in an expanded configuration has a frame height-to-heart valve diameter ratio (e.g., FTVD ratio) of 1.01 to 1.1, 1.1 to 1.2, 1.2 to 1.3, 1.3 to 1.4, 1.4 to 1.5, 1.5 to 1.6, 1.6 to 1.7, 1.7 to 1.8, 1.8 to 1.9, 1.9 to 2.0, 2.0 to 2.5, 2.5 to 3.0, 3.0 to 4.0, 4.0 to 5.0, 5.0 to 6.0, 6.0 to 7.0, 7.0 to 8.0, 8.0 to 9.0, 9.0 to 10.0, or more than 10.0. In some cases, the height 137 of a frame structure 12 of a valve prosthesis 10F in an expanded configuration is 5%, 10%, 15%, 20%, 25%, 30%, 35%, 40%, 45%, 50%, 60%, 70%, 80%, 90%, 100%, 110%, 120%, 130%, 140%, 150%, 160%, 170%, 180%, 190%, 200%, 250%, 300%, 400%, 500%, 600%, 700%, 800%, 900%, or 1000% larger than the diameter of a heart valve in which the valve prosthesis 10F is deployed. In some cases, the height 137 of a frame structure 12 of a valve prosthesis 10F in an expanded configuration is at least 5%, 10%, 15%, 20%, 25%, 30%, 35%, 40%, 45%, 50%, 60%, 70%, 80%, 90%, 100%, 110%, 120%, 130%, 140%, 150%, 160%, 170%, 180%, 190%, 200%, 250%, 300%, 400%, 500%, 600%, 700%, 800%, 900%, or 1000% larger than the diameter of a heart valve in which the valve prosthesis 10F is deployed. In some cases, the height 137 of a frame structure 12 of a valve prosthesis 10F in an expanded configuration is at most 5%, 10%, 15%, 20%, 25%, 30%, 35%, 40%, 45%, 50%, 60%, 70%, 80%, 90%, 100%, 110%, 120%, 130%, 140%, 150%, 160%, 170%, 180%, 190%, 200%, 250%, 300%, 400%, 500%, 600%, 700%, 800%, 900%, or 1000% larger than the diameter of a heart valve in which the valve prosthesis 10F is deployed.

In some cases, the height 137 of a frame structure 12 of an expanded valve prosthesis 10F is equal to the diameter of a heart valve in which the valve prosthesis 10F is deployed (e.g., a FTVD ratio equal to 1). In some cases, the height 137 of a frame structure 12 of an expanded valve prosthesis 10F is less than the diameter of a heart valve in which the valve prosthesis 10F is deployed (e.g., a FTVD ratio less than 1). In some cases, the height 137 of a frame structure 12 of a valve prosthesis 10F in an expanded configuration is 1% larger than to 1000% larger than the diameter of a heart valve in which the valve prosthesis 10F is deployed. In some cases, the height 137 of a frame structure 12 of a valve prosthesis 10F in an expanded configuration is 1% to 5%, 5% to 10%, 5% to 15%, 5% to 20%, 5% to 25%, 5% to 30%, 5% to 35%, 5% to 40%, 5% to 45%, 5% to 50%, 5% to 60%, 5% to 70%, 10% to 15%, 10% to 20%, 10% to 25%, 10% to 30%, 10% to 35%, 10% to 40%, 10% to 45%, 10% to 50%, 10% to 60%, 10% to 70%, 15% to 20%, 15% to 25%, 15% to 30%, 15% to 35%, 15% to 40%, 15% to 45%, 15% to 50%, 15% to 60%, 15% to 70%, 20% to 25%, 20% to 30%, 20% to 35%, 20% to 40%, 20% to 45%, 20% to 50%, 20% to 60%, 20% to 70%, 25% to 30%, 25% to 35%, 25% to 40%, 25% to 45%, 25% to 50%, 25% to 60%, 25% to 70%, 30% to 35%, 30% to 40%, 30% to 45%, 30% to 50%, 30% to 60%, 30% to 70%, 35% to 40%, 35% to 45%, 35% to 50%, 35% to 60%, 35% to 70%, 40% to 45%, 40% to 50%, 40% to 60%, 40% to 70%, 45% to 50%, 45% to 60%, 45% to 70%, 50% to 60%, 50% to 70%, 60% to 70%, 70% to 80%, 80% to 90%, 90% to 100%, 100% to 110%, 110% to 120%, 120% to 130%, 130% to 140%, 140% to 150%, 150% to 160%, 160% to 170%, 170% to 180%, 180% to 190%, 190% to 200%, 200% to 250%, 250% to 300%, 300% to 400%, 400% to 500%, 500% to 600%, 600% to 700%, 700% to 800%, 800% to 900%, 900% to 1000%, or over 1000% less than the diameter of a heart valve in which the device is deployed. In some cases, a valve prosthesis 10F in an expanded configuration has a frame height-to-heart valve diameter ratio (e.g., FTVD ratio) of 0.99 to 0.90, 0.90 to 0.80, 0.80 to 0.70, 0.70 to 0.60, 0.60 to 0.50, 0.50 to 0.40, 0.40 to 0.30, 0.30 to 0.25, 0.25 to 0.20, 0.20 to 0.15, 0.15 to 0.10, 0.10 to 0.01, less than 0.01, or between 1.00 and 0.99. In some cases, the height 137 of a frame structure 12 of a valve prosthesis 10F in an expanded configuration is 5%, 10%, 15%, 20%, 25%, 30%, 35%, 40%, 45%, 50%, 60%, 70%, 80%, 90%, 100%, 110%, 120%, 130%, 140%, 150%, 160%, 170%, 180%, 190%, 200%, 250%, 300%, 400%, 500%, 600%, 700%, 800%, 900%, or 1000% less than the diameter of a heart valve in which the device is deployed. In some cases, the height 137 of a frame structure 12 of a valve prosthesis 10F in an expanded configuration is at least 5%, 10%, 15%, 20%, 25%, 30%, 35%, 40%, 45%, 50%, 60%, 70%, 80%, 90%, 100%, 110%, 120%, 130%, 140%, 150%, 160%, 170%, 180%, 190%, 200%, 250%, 300%, 400%, 500%, 600%, 700%, 800%, 900%, or 1000% smaller than the diameter of a heart valve in which the valve prosthesis 10F is deployed. In some cases, the height 137 of a frame structure 12 of a valve prosthesis 10F in an expanded configuration is at most 5%, 10%, 15%, 20%, 25%, 30%, 35%, 40%, 45%, 50%, 60%, 70%, 80%, 90%, 100%, 110%, 120%, 130%, 140%, 150%, 160%, 170%, 180%, 190%, 200%, 250%, 300%, 400%, 500%, 600%, 700%, 800%, 900%, or 1000% smaller than the diameter of a heart valve in which the valve prosthesis 10F is deployed.

As described herein, minimizing the size of a frame body of a valve prosthesis 10 (or any valve prosthesis described herein) can be advantageous to the function and/or manufacturing of the valve prosthesis 10. For example, minimizing the height 137 of a frame body of a valve prosthesis 10 (e.g., in an expanded configuration as shown in FIG. 13A, FIG. 14, or FIG. 16A, for example, relative to the frame shown in FIG. 13B) can be advantageous to the function and/or manufacturing of the valve prosthesis 10. Minimizing the frame height of valve prosthesis 10 can minimize obstruction of fluid flow in the heart of a subject, reduce immunogenicity of valve prosthesis device (e.g., by reducing the surface area of metal comprising the valve prosthesis device), and/or reduce cost of valve prosthesis 10 manufacture.

In some cases, a frame height 137 of a valve prosthesis 10 (e.g., a valve prosthesis device having a minimal frame body size) is 5 millimeters to 26 millimeters. In some cases, a frame height 137 of a valve prosthesis 10 (e.g., a valve prosthesis device having a minimal frame body size) is 5 millimeters to 7 millimeters, 5 millimeters to 8 millimeters, 5 millimeters to 9 millimeters, 5 millimeters to 10 millimeters, 5 millimeters to 10.5 millimeters, 5 millimeters to 11 millimeters, 5 millimeters to 12 millimeters, 5 millimeters to 13.5 millimeters, 5 millimeters to 15 millimeters, 5 millimeters to 20 millimeters, 5 millimeters to 26 millimeters, 7 millimeters to 8 millimeters, 7 millimeters to 9 millimeters, 7 millimeters to 10 millimeters, 7 millimeters to 10.5 millimeters, 7 millimeters to 11 millimeters, 7 millimeters to 12 millimeters, 7 millimeters to 13.5 millimeters, 7 millimeters to 15 millimeters, 7 millimeters to 20 millimeters, 7 millimeters to 26 millimeters, 8 millimeters to 9 millimeters, 8 millimeters to 10 millimeters, 8 millimeters to 10.5 millimeters, 8 millimeters to 11 millimeters, 8 millimeters to 12 millimeters, 8 millimeters to 13.5 millimeters, 8 millimeters to 15 millimeters, 8 millimeters to 20 millimeters, 8 millimeters to 26 millimeters, 9 millimeters to 10 millimeters, 9 millimeters to 10.5 millimeters, 9 millimeters to 11 millimeters, 9 millimeters to 12 millimeters, 9 millimeters to 13.5 millimeters, 9 millimeters to 15 millimeters, 9 millimeters to 20 millimeters, 9 millimeters to 26 millimeters, 10 millimeters to 10.5 millimeters, 10 millimeters to 11 millimeters, 10 millimeters to 12 millimeters, 10 millimeters to 13.5 millimeters, 10 millimeters to 15 millimeters, 10 millimeters to 20 millimeters, 10 millimeters to 26 millimeters, 10.5 millimeters to 11 millimeters, 10.5 millimeters to 12 millimeters, 10.5 millimeters to 13.5 millimeters, 10.5 millimeters to 15 millimeters, 10.5 millimeters to 20 millimeters, 10.5 millimeters to 26 millimeters, 11 millimeters to 12 millimeters, 11 millimeters to 13.5 millimeters, 11 millimeters to 15 millimeters, 11 millimeters to 20 millimeters, 11 millimeters to 26 millimeters, 12 millimeters to 13.5 millimeters, 12 millimeters to 15 millimeters, 12 millimeters to 20 millimeters, 12 millimeters to 26 millimeters, 13.5 millimeters to 15 millimeters, 13.5 millimeters to 20 millimeters, 13.5 millimeters to 26 millimeters, 15 millimeters to 20 millimeters, 15 millimeters to 26 millimeters, or 20 millimeters to 26 millimeters. In some cases, a frame height 137 of a valve prosthesis 10 (e.g., a valve prosthesis device having a minimal frame body size) is 5 millimeters, 7 millimeters, 8 millimeters, 9 millimeters, 10 millimeters, 10.5 millimeters, 11 millimeters, 12 millimeters, 13.5 millimeters, 15 millimeters, 20 millimeters, or 26 millimeters. In some cases, a frame height 137 of a valve prosthesis 10 (e.g., a valve prosthesis device having a minimal frame body size) is at least 5 millimeters, 7 millimeters, 8 millimeters, 9 millimeters, 10 millimeters, 10.5 millimeters, 11 millimeters, 12 millimeters, 13.5 millimeters, 15 millimeters, or 20 millimeters. In some cases, a frame height 137 of a valve prosthesis 10 (e.g., a valve prosthesis device having a minimal frame body size) is at most 7 millimeters, 8 millimeters, 9 millimeters, 10 millimeters, 10.5 millimeters, 11 millimeters, 12 millimeters, 13.5 millimeters, 15 millimeters, 20 millimeters, or 26 millimeters.

Some embodiments of a valve prosthesis 10 disclosed herein (e.g., for mitral valve replacement) having a minimal frame body size (e.g., a minimized frame height 137) have a frame height 137 of 13 millimeters to 38 millimeters. In some cases, a valve prosthesis 10 having a minimal frame body size (e.g., for replacement of a mitral valve) can have a frame height 137 of 13 millimeters to 15.5 millimeters, 13 millimeters to 18 millimeters, 13 millimeters to 20 millimeters, 13 millimeters to 22 millimeters, 13 millimeters to 24 millimeters, 13 millimeters to 26 millimeters, 13 millimeters to 28 millimeters, 13 millimeters to 30 millimeters, 13 millimeters to 32 millimeters, 13 millimeters to 35 millimeters, 13 millimeters to 38 millimeters, 15.5 millimeters to 18 millimeters, 15.5 millimeters to 20 millimeters, 15.5 millimeters to 22 millimeters, 15.5 millimeters to 24 millimeters, 15.5 millimeters to 26 millimeters, 15.5 millimeters to 28 millimeters, 15.5 millimeters to 30 millimeters, 15.5 millimeters to 32 millimeters, 15.5 millimeters to 35 millimeters, 15.5 millimeters to 38 millimeters, 18 millimeters to 20 millimeters, 18 millimeters to 22 millimeters, 18 millimeters to 24 millimeters, 18 millimeters to 26 millimeters, 18 millimeters to 28 millimeters, 18 millimeters to 30 millimeters, 18 millimeters to 32 millimeters, 18 millimeters to 35 millimeters, 18 millimeters to 38 millimeters, 20 millimeters to 22 millimeters, 20 millimeters to 24 millimeters, 20 millimeters to 26 millimeters, 20 millimeters to 28 millimeters, 20 millimeters to 30 millimeters, 20 millimeters to 32 millimeters, 20 millimeters to 35 millimeters, 20 millimeters to 38 millimeters, 22 millimeters to 24 millimeters, 22 millimeters to 26 millimeters, 22 millimeters to 28 millimeters, 22 millimeters to 30 millimeters, 22 millimeters to 32 millimeters, 22 millimeters to 35 millimeters, 22 millimeters to 38 millimeters, 24 millimeters to 26 millimeters, 24 millimeters to 28 millimeters, 24 millimeters to 30 millimeters, 24 millimeters to 32 millimeters, 24 millimeters to 35 millimeters, 24 millimeters to 38 millimeters, 26 millimeters to 28 millimeters, 26 millimeters to 30 millimeters, 26 millimeters to 32 millimeters, 26 millimeters to 35 millimeters, 26 millimeters to 38 millimeters, 28 millimeters to 30 millimeters, 28 millimeters to 32 millimeters, 28 millimeters to 35 millimeters, 28 millimeters to 38 millimeters, 30 millimeters to 32 millimeters, 30 millimeters to 35 millimeters, 30 millimeters to 38 millimeters, 32 millimeters to 35 millimeters, 32 millimeters to 38 millimeters, or 35 millimeters to 38 millimeters. In some cases, a valve prosthesis 10 having a minimal frame body size (e.g., for replacement of a mitral valve) can have a frame height 137 of 13 millimeters, 15.5 millimeters, 18 millimeters, 20 millimeters, 22 millimeters, 24 millimeters, 26 millimeters, 28 millimeters, 30 millimeters, 32 millimeters, 35 millimeters, or 38 millimeters. In some cases, a valve prosthesis 10 having a minimal frame body size (e.g., for replacement of a mitral valve) can have a frame height 137 of at least 13 millimeters, 15.5 millimeters, 18 millimeters, 20 millimeters, 22 millimeters, 24 millimeters, 26 millimeters, 28 millimeters, 30 millimeters, 32 millimeters, or 35 millimeters. In some cases, a valve prosthesis 10 having a minimal frame body size (e.g., for replacement of a mitral valve) can have a frame height 137 of at most 15.5 millimeters, 18 millimeters, 20 millimeters, 22 millimeters, 24 millimeters, 26 millimeters, 28 millimeters, 30 millimeters, 32 millimeters, 35 millimeters, or 38 millimeters.

Some embodiments of a valve prosthesis 10 disclosed herein (e.g., for tricuspid valve replacement) having a minimal frame body size (e.g., a minimized frame height 137) have a frame height 137 of 15 millimeters to 45 millimeters. In some cases, a valve prosthesis 10 having a minimal frame body size (e.g., for replacement of a tricuspid valve) can have a frame height 137 of 15 millimeters to 17.5 millimeters, 15 millimeters to 19 millimeters, 15 millimeters to 20.5 millimeters, 15 millimeters to 21.5 millimeters, 15 millimeters to 23 millimeters, 15 millimeters to 24.5 millimeters, 15 millimeters to 27 millimeters, 15 millimeters to 30 millimeters, 15 millimeters to 33 millimeters, 15 millimeters to 39 millimeters, 15 millimeters to 45 millimeters, 17.5 millimeters to 19 millimeters, 17.5 millimeters to 20.5 millimeters, 17.5 millimeters to 21.5 millimeters, 17.5 millimeters to 23 millimeters, 17.5 millimeters to 24.5 millimeters, 17.5 millimeters to 27 millimeters, 17.5 millimeters to 30 millimeters, 17.5 millimeters to 33 millimeters, 17.5 millimeters to 39 millimeters, 17.5 millimeters to 45 millimeters, 19 millimeters to 20.5 millimeters, 19 millimeters to 21.5 millimeters, 19 millimeters to 23 millimeters, 19 millimeters to 24.5 millimeters, 19 millimeters to 27 millimeters, 19 millimeters to 30 millimeters, 19 millimeters to 33 millimeters, 19 millimeters to 39 millimeters, 19 millimeters to 45 millimeters, 20.5 millimeters to 21.5 millimeters, 20.5 millimeters to 23 millimeters, 20.5 millimeters to 24.5 millimeters, 20.5 millimeters to 27 millimeters, 20.5 millimeters to 30 millimeters, 20.5 millimeters to 33 millimeters, 20.5 millimeters to 39 millimeters, 20.5 millimeters to 45 millimeters, 21.5 millimeters to 23 millimeters, 21.5 millimeters to 24.5 millimeters, 21.5 millimeters to 27 millimeters, 21.5 millimeters to 30 millimeters, 21.5 millimeters to 33 millimeters, 21.5 millimeters to 39 millimeters, 21.5 millimeters to 45 millimeters, 23 millimeters to 24.5 millimeters, 23 millimeters to 27 millimeters, 23 millimeters to 30 millimeters, 23 millimeters to 33 millimeters, 23 millimeters to 39 millimeters, 23 millimeters to 45 millimeters, 24.5 millimeters to 27 millimeters, 24.5 millimeters to 30 millimeters, 24.5 millimeters to 33 millimeters, 24.5 millimeters to 39 millimeters, 24.5 millimeters to 45 millimeters, 27 millimeters to 30 millimeters, 27 millimeters to 33 millimeters, 27 millimeters to 39 millimeters, 27 millimeters to 45 millimeters, 30 millimeters to 33 millimeters, 30 millimeters to 39 millimeters, 30 millimeters to 45 millimeters, 33 millimeters to 39 millimeters, 33 millimeters to 45 millimeters, or 39 millimeters to 45 millimeters. In some cases, a valve prosthesis 10 having a minimal frame body size (e.g., for replacement of a tricuspid valve) can have a frame height 137 of 15 millimeters, 17.5 millimeters, 19 millimeters, 20.5 millimeters, 21.5 millimeters, 23 millimeters, 24.5 millimeters, 27 millimeters, 30 millimeters, 33 millimeters, 39 millimeters, or 45 millimeters. In some cases, a valve prosthesis 10 having a minimal frame body size (e.g., for replacement of a tricuspid valve) can have a frame height 137 of at least 15 millimeters, 17.5 millimeters, 19 millimeters, 20.5 millimeters, 21.5 millimeters, 23 millimeters, 24.5 millimeters, 27 millimeters, 30 millimeters, 33 millimeters, or 39 millimeters. In some cases, a valve prosthesis 10 having a minimal frame body size (e.g., for replacement of a tricuspid valve) can have a frame height 137 of at most 17.5 millimeters, 19 millimeters, 20.5 millimeters, 21.5 millimeters, 23 millimeters, 24.5 millimeters, 27 millimeters, 30 millimeters, 33 millimeters, 39 millimeters, or 45 millimeters.

Some embodiments of a valve prosthesis 10 disclosed herein (e.g., for aortic valve replacement) having a minimal frame body size (e.g., a minimized frame height 137) have a frame height 137 of 9.5 millimeters to 27.5 millimeters. In some cases, a valve prosthesis 10 having a minimal frame body size (e.g., for replacement of an aortic valve) can have a frame height 137 of 9.5 millimeters to 11 millimeters, 9.5 millimeters to 12 millimeters, 9.5 millimeters to 14 millimeters, 9.5 millimeters to 15 millimeters, 9.5 millimeters to 16 millimeters, 9.5 millimeters to 18 millimeters, 9.5 millimeters to 20 millimeters, 9.5 millimeters to 22 millimeters, 9.5 millimeters to 24 millimeters, 9.5 millimeters to 26 millimeters, 9.5 millimeters to 27.5 millimeters, 11 millimeters to 12 millimeters, 11 millimeters to 14 millimeters, 11 millimeters to 15 millimeters, 11 millimeters to 16 millimeters, 11 millimeters to 18 millimeters, 11 millimeters to 20 millimeters, 11 millimeters to 22 millimeters, 11 millimeters to 24 millimeters, 11 millimeters to 26 millimeters, 11 millimeters to 27.5 millimeters, 12 millimeters to 14 millimeters, 12 millimeters to 15 millimeters, 12 millimeters to 16 millimeters, 12 millimeters to 18 millimeters, 12 millimeters to 20 millimeters, 12 millimeters to 22 millimeters, 12 millimeters to 24 millimeters, 12 millimeters to 26 millimeters, 12 millimeters to 27.5 millimeters, 14 millimeters to 15 millimeters, 14 millimeters to 16 millimeters, 14 millimeters to 18 millimeters, 14 millimeters to 20 millimeters, 14 millimeters to 22 millimeters, 14 millimeters to 24 millimeters, 14 millimeters to 26 millimeters, 14 millimeters to 27.5 millimeters, 15 millimeters to 16 millimeters, 15 millimeters to 18 millimeters, 15 millimeters to 20 millimeters, 15 millimeters to 22 millimeters, 15 millimeters to 24 millimeters, 15 millimeters to 26 millimeters, 15 millimeters to 27.5 millimeters, 16 millimeters to 18 millimeters, 16 millimeters to 20 millimeters, 16 millimeters to 22 millimeters, 16 millimeters to 24 millimeters, 16 millimeters to 26 millimeters, 16 millimeters to 27.5 millimeters, 18 millimeters to 20 millimeters, 18 millimeters to 22 millimeters, 18 millimeters to 24 millimeters, 18 millimeters to 26 millimeters, 18 millimeters to 27.5 millimeters, 20 millimeters to 22 millimeters, 20 millimeters to 24 millimeters, 20 millimeters to 26 millimeters, 20 millimeters to 27.5 millimeters, 22 millimeters to 24 millimeters, 22 millimeters to 26 millimeters, 22 millimeters to 27.5 millimeters, 24 millimeters to 26 millimeters, 24 millimeters to 27.5 millimeters, or 26 millimeters to 27.5 millimeters. In some cases, a valve prosthesis 10 having a minimal frame body size (e.g., for replacement of an aortic valve) can have a frame height 137 of 9.5 millimeters, 11 millimeters, 12 millimeters, 14 millimeters, 15 millimeters, 16 millimeters, 18 millimeters, 20 millimeters, 22 millimeters, 24 millimeters, 26 millimeters, or 27.5 millimeters. In some cases, a valve prosthesis 10 having a minimal frame body size (e.g., for replacement of an aortic valve) can have a frame height 137 of at least 9.5 millimeters, 11 millimeters, 12 millimeters, 14 millimeters, 15 millimeters, 16 millimeters, 18 millimeters, 20 millimeters, 22 millimeters, 24 millimeters, or 26 millimeters. In some cases, a valve prosthesis 10 having a minimal frame body size (e.g., for replacement of an aortic valve) can have a frame height 137 of at most 11 millimeters, 12 millimeters, 14 millimeters, 15 millimeters, 16 millimeters, 18 millimeters, 20 millimeters, 22 millimeters, 24 millimeters, 26 millimeters, or 27.5 millimeters.

Some embodiments of a valve prosthesis 10 disclosed herein (e.g., for pulmonary valve replacement) having a minimal frame body size (e.g., a minimized frame height 137) have a frame height 137 of 10 millimeters to 28 millimeters. In some cases, a valve prosthesis 10 having a minimal frame body size (e.g., for replacement of a pulmonary valve) can have a frame height 137 of 10 millimeters to 11 millimeters, 10 millimeters to 12 millimeters, 10 millimeters to 14 millimeters, 10 millimeters to 15 millimeters, 10 millimeters to 16 millimeters, 10 millimeters to 18 millimeters, 10 millimeters to 20 millimeters, 10 millimeters to 22 millimeters, 10 millimeters to 24 millimeters, 10 millimeters to 26 millimeters, 10 millimeters to 28 millimeters, 11 millimeters to 12 millimeters, 11 millimeters to 14 millimeters, 11 millimeters to 15 millimeters, 11 millimeters to 16 millimeters, 11 millimeters to 18 millimeters, 11 millimeters to 20 millimeters, 11 millimeters to 22 millimeters, 11 millimeters to 24 millimeters, 11 millimeters to 26 millimeters, 11 millimeters to 28 millimeters, 12 millimeters to 14 millimeters, 12 millimeters to 15 millimeters, 12 millimeters to 16 millimeters, 12 millimeters to 18 millimeters, 12 millimeters to 20 millimeters, 12 millimeters to 22 millimeters, 12 millimeters to 24 millimeters, 12 millimeters to 26 millimeters, 12 millimeters to 28 millimeters, 14 millimeters to 15 millimeters, 14 millimeters to 16 millimeters, 14 millimeters to 18 millimeters, 14 millimeters to 20 millimeters, 14 millimeters to 22 millimeters, 14 millimeters to 24 millimeters, 14 millimeters to 26 millimeters, 14 millimeters to 28 millimeters, 15 millimeters to 16 millimeters, 15 millimeters to 18 millimeters, 15 millimeters to 20 millimeters, 15 millimeters to 22 millimeters, 15 millimeters to 24 millimeters, 15 millimeters to 26 millimeters, 15 millimeters to 28 millimeters, 16 millimeters to 18 millimeters, 16 millimeters to 20 millimeters, 16 millimeters to 22 millimeters, 16 millimeters to 24 millimeters, 16 millimeters to 26 millimeters, 16 millimeters to 28 millimeters, 18 millimeters to 20 millimeters, 18 millimeters to 22 millimeters, 18 millimeters to 24 millimeters, 18 millimeters to 26 millimeters, 18 millimeters to 28 millimeters, 20 millimeters to 22 millimeters, 20 millimeters to 24 millimeters, 20 millimeters to 26 millimeters, 20 millimeters to 28 millimeters, 22 millimeters to 24 millimeters, 22 millimeters to 26 millimeters, 22 millimeters to 28 millimeters, 24 millimeters to 26 millimeters, 24 millimeters to 28 millimeters, or 26 millimeters to 28 millimeters. In some cases, a valve prosthesis 10 having a minimal frame body size (e.g., for replacement of a pulmonary valve) can have a frame height 137 of 10 millimeters, 11 millimeters, 12 millimeters, 14 millimeters, 15 millimeters, 16 millimeters, 18 millimeters, 20 millimeters, 22 millimeters, 24 millimeters, 26 millimeters, or 28 millimeters. In some cases, a valve prosthesis 10 having a minimal frame body size (e.g., for replacement of a pulmonary valve) can have a frame height 137 of at least 10 millimeters, 11 millimeters, 12 millimeters, 14 millimeters, 15 millimeters, 16 millimeters, 18 millimeters, 20 millimeters, 22 millimeters, 24 millimeters, or 26 millimeters. In some cases, a valve prosthesis 10 having a minimal frame body size (e.g., for replacement of a pulmonary valve) can have a frame height 137 of at most 11 millimeters, 12 millimeters, 14 millimeters, 15 millimeters, 16 millimeters, 18 millimeters, 20 millimeters, 22 millimeters, 24 millimeters, 26 millimeters, or 28 millimeters.

In some cases, a frame height 137 of a valve prosthesis 10 having a diameter of from 15.0 millimeters to 30 millimeters is 5 millimeters to 26 millimeters. In some cases, a frame height 137 of a valve prosthesis 10 having a diameter of from 15.0 millimeters to 30 millimeters is 5 millimeters to 7 millimeters, 5 millimeters to 8 millimeters, 5 millimeters to 9 millimeters, 5 millimeters to 10 millimeters, 5 millimeters to 10.5 millimeters, 5 millimeters to 11 millimeters, 5 millimeters to 12 millimeters, 5 millimeters to 13.5 millimeters, 5 millimeters to 15 millimeters, 5 millimeters to 20 millimeters, 5 millimeters to 26 millimeters, 7 millimeters to 8 millimeters, 7 millimeters to 9 millimeters, 7 millimeters to 10 millimeters, 7 millimeters to 10.5 millimeters, 7 millimeters to 11 millimeters, 7 millimeters to 12 millimeters, 7 millimeters to 13.5 millimeters, 7 millimeters to 15 millimeters, 7 millimeters to 20 millimeters, 7 millimeters to 26 millimeters, 8 millimeters to 9 millimeters, 8 millimeters to 10 millimeters, 8 millimeters to 10.5 millimeters, 8 millimeters to 11 millimeters, 8 millimeters to 12 millimeters, 8 millimeters to 13.5 millimeters, 8 millimeters to 15 millimeters, 8 millimeters to 20 millimeters, 8 millimeters to 26 millimeters, 9 millimeters to 10 millimeters, 9 millimeters to 10.5 millimeters, 9 millimeters to 11 millimeters, 9 millimeters to 12 millimeters, 9 millimeters to 13.5 millimeters, 9 millimeters to 15 millimeters, 9 millimeters to 20 millimeters, 9 millimeters to 26 millimeters, 10 millimeters to 10.5 millimeters, 10 millimeters to 11 millimeters, 10 millimeters to 12 millimeters, 10 millimeters to 13.5 millimeters, 10 millimeters to 15 millimeters, 10 millimeters to 20 millimeters, 10 millimeters to 26 millimeters, 10.5 millimeters to 11 millimeters, 10.5 millimeters to 12 millimeters, 10.5 millimeters to 13.5 millimeters, 10.5 millimeters to 15 millimeters, 10.5 millimeters to 20 millimeters, 10.5 millimeters to 26 millimeters, 11 millimeters to 12 millimeters, 11 millimeters to 13.5 millimeters, 11 millimeters to 15 millimeters, 11 millimeters to 20 millimeters, 11 millimeters to 26 millimeters, 12 millimeters to 13.5 millimeters, 12 millimeters to 15 millimeters, 12 millimeters to 20 millimeters, 12 millimeters to 26 millimeters, 13.5 millimeters to 15 millimeters, 13.5 millimeters to 20 millimeters, 13.5 millimeters to 26 millimeters, 16 millimeters to 17.5 millimeters, 15 millimeters to 20 millimeters, 15 millimeters to 26 millimeters, 21.0 millimeters to 26.0 millimeters, or 20 millimeters to 26 millimeters. In some cases, a frame height 137 of a valve prosthesis 10 having a diameter of from 15.0 millimeters to 30 millimeters is 5 millimeters, 7 millimeters, 8 millimeters, 9 millimeters, 10 millimeters, 10.5 millimeters, 11 millimeters, 12 millimeters, 13.5 millimeters, 15 millimeters, 20 millimeters, or 26 millimeters. In some cases, a frame height 137 of a valve prosthesis 10 (e.g., a valve prosthesis device having a minimal frame body size) is at least 5 millimeters, 7 millimeters, 8 millimeters, 9 millimeters, 10 millimeters, 10.5 millimeters, 11 millimeters, 12 millimeters, 13.5 millimeters, 15 millimeters, or 20 millimeters. In some cases, a frame height 137 of a valve prosthesis 10 having a diameter of from 15.0 millimeters to 30 millimeters is at most 7 millimeters, 8 millimeters, 9 millimeters, 10 millimeters, 10.5 millimeters, 11 millimeters, 12 millimeters, 13.5 millimeters, 15 millimeters, 20 millimeters, or 26 millimeters.

Some embodiments of a valve prosthesis 10 disclosed herein having a minimal frame body size (e.g., a minimized frame height) have a frame height 137 of 0.05 times to 2 times the magnitude of a diameter 139 of the frame of the valve prosthesis device 10. In some cases, a valve prosthesis 10 disclosed herein can have a minimal frame body size has a frame height 137 of 0.05 times to 0.25 times, 0.05 times to 0.4 times, 0.05 times to 0.5 times, 0.05 times to 0.55 times, 0.05 times to 0.6 times, 0.05 times to 0.65 times, 0.05 times to 0.7 times, 0.05 times to 0.8 times, 0.05 times to 1 time, 0.05 times to 1.5 times, 0.05 times to 2 times, 0.25 times to 0.4 times, 0.25 times to 0.5 times, 0.25 times to 0.55 times, 0.25 times to 0.6 times, 0.25 times to 0.65 times, 0.25 times to 0.7 times, 0.25 times to 0.8 times, 0.25 times to 1 time, 0.25 times to 1.5 times, 0.25 times to 2 times, 0.4 times to 0.5 times, 0.4 times to 0.55 times, 0.4 times to 0.6 times, 0.4 times to 0.65 times, 0.4 times to 0.7 times, 0.4 times to 0.8 times, 0.4 times to 1 time, 0.4 times to 1.5 times, 0.4 times to 2 times, 0.5 times to 0.55 times, 0.5 times to 0.6 times, 0.5 times to 0.65 times, 0.5 times to 0.7 times, 0.5 times to 0.8 times, 0.5 times to 1 time, 0.5 times to 1.5 times, 0.5 times to 2 times, 0.55 times to 0.6 times, 0.55 times to 0.65 times, 0.55 times to 0.7 times, 0.55 times to 0.8 times, 0.55 times to 1 time, 0.55 times to 1.5 times, 0.55 times to 2 times, 0.6 times to 0.65 times, 0.6 times to 0.7 times, 0.6 times to 0.8 times, 0.6 times to 1 time, 0.6 times to 1.5 times, 0.6 times to 2 times, 0.65 times to 0.7 times, 0.65 times to 0.8 times, 0.65 times to 1 time, 0.65 times to 1.5 times, 0.65 times to 2 times, 0.7 times to 0.8 times, 0.7 times to 1 time, 0.7 times to 1.5 times, 0.7 times to 2 times, 0.8 times to 1 time, 0.8 times to 1.5 times, 0.8 times to 2 times, 1 time to 1.5 times, 1 time to 2 times, or 1.5 times to 2 times the magnitude of a diameter 139 of the frame of the valve prosthesis device 10. In some cases, a valve prosthesis 10 disclosed herein can have a minimal frame body size has a frame height 137 of 0.05 times, 0.25 times, 0.4 times, 0.5 times, 0.55 times, 0.6 times, 0.65 times, 0.7 times, 0.8 times, 1 time, 1.5 times, or 2 times the magnitude of a diameter 139 of the frame of the valve prosthesis device 10. In some cases, a valve prosthesis 10 disclosed herein can have a minimal frame body size has a frame height 137 of at least 0.05 times, 0.25 times, 0.4 times, 0.5 times, 0.55 times, 0.6 times, 0.65 times, 0.7 times, 0.8 times, 1 time, or 1.5 times the magnitude of a diameter 139 of the frame of the valve prosthesis device 10. In some cases, a valve prosthesis 10 disclosed herein can have a minimal frame body size has a frame height 137 of at most 0.25 times, 0.4 times, 0.5 times, 0.55 times, 0.6 times, 0.65 times, 0.7 times, 0.8 times, 1 time, 1.5 times, or 2 times the magnitude of a diameter 139 of the frame of the valve prosthesis device 10.

Some embodiments of a valve prosthesis 10 disclosed herein having a minimal frame body size (e.g., a minimized frame height) have a frame height 137 of 1.1 times to 2.5 times the annular height of a heart valve. In some cases, a valve prosthesis 10 having a minimal frame body size can have a frame height 137 of 1.1 times to 1.2 times, 1.1 times to 1.3 times, 1.1 times to 1.4 times, 1.1 times to 1.5 times, 1.1 times to 1.6 times, 1.1 times to 1.7 times, 1.1 times to 1.8 times, 1.1 times to 1.9 times, 1.1 times to 2 times, 1.1 times to 2.3 times, 1.1 times to 2.5 times, 1.2 times to 1.3 times, 1.2 times to 1.4 times, 1.2 times to 1.5 times, 1.2 times to 1.6 times, 1.2 times to 1.7 times, 1.2 times to 1.8 times, 1.2 times to 1.9 times, 1.2 times to 2 times, 1.2 times to 2.3 times, 1.2 times to 2.5 times, 1.3 times to 1.4 times, 1.3 times to 1.5 times, 1.3 times to 1.6 times, 1.3 times to 1.7 times, 1.3 times to 1.8 times, 1.3 times to 1.9 times, 1.3 times to 2 times, 1.3 times to 2.3 times, 1.3 times to 2.5 times, 1.4 times to 1.5 times, 1.4 times to 1.6 times, 1.4 times to 1.7 times, 1.4 times to 1.8 times, 1.4 times to 1.9 times, 1.4 times to 2 times, 1.4 times to 2.3 times, 1.4 times to 2.5 times, 1.5 times to 1.6 times, 1.5 times to 1.7 times, 1.5 times to 1.8 times, 1.5 times to 1.9 times, 1.5 times to 2 times, 1.5 times to 2.3 times, 1.5 times to 2.5 times, 1.6 times to 1.7 times, 1.6 times to 1.8 times, 1.6 times to 1.9 times, 1.6 times to 2 times, 1.6 times to 2.3 times, 1.6 times to 2.5 times, 1.7 times to 1.8 times, 1.7 times to 1.9 times, 1.7 times to 2 times, 1.7 times to 2.3 times, 1.7 times to 2.5 times, 1.8 times to 1.9 times, 1.8 times to 2 times, 1.8 times to 2.3 times, 1.8 times to 2.5 times, 1.9 times to 2 times, 1.9 times to 2.3 times, 1.9 times to 2.5 times, 2 times to 2.3 times, 2 times to 2.5 times, or 2.3 times to 2.5 times the annular height of a heart valve. In some cases, a valve prosthesis 10 having a minimal frame body size can have a frame height 137 of 1.1 times, 1.2 times, 1.3 times, 1.4 times, 1.5 times, 1.6 times, 1.7 times, 1.8 times, 1.9 times, 2 times, 2.3 times, or 2.5 times the annular height of a heart valve. In some cases, a valve prosthesis 10 having a minimal frame body size can have a frame height 137 of at least 1.1 times, 1.2 times, 1.3 times, 1.4 times, 1.5 times, 1.6 times, 1.7 times, 1.8 times, 1.9 times, 2 times, or 2.3 times the annular height of a heart valve. In some cases, a valve prosthesis 10 having a minimal frame body size can have a frame height 137 of at most 1.2 times, 1.3 times, 1.4 times, 1.5 times, 1.6 times, 1.7 times, 1.8 times, 1.9 times, 2 times, 2.3 times, or 2.5 times the annular height of a heart valve.

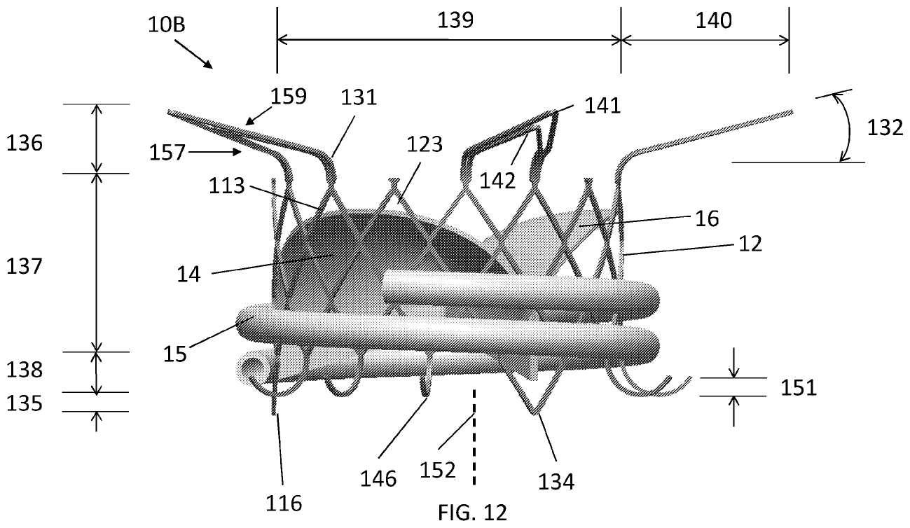

In some cases, a frame body height 137 is measured from a distal end of lattice structure of frame structure 12 to a proximal end of lattice structure of frame structure 12 (e.g., as shown in FIG. 12). In some cases, a frame body height 137 is measured from a proximal end of a longitudinal strut 113 of frame structure 12 to a distal end of the longitudinal strut (e.g., as shown in FIG. 16A). In some cases, frame body height 137 is measured from a proximal end of the longest longitudinal strut 113 of frame structure 12 to a distal end of same longitudinal strut 133.

In some cases, frame structure 12 has a total height 237 that is the sum of the frame body height 137 and one or more of flange height 136, distance 138 (e.g., as shown in FIG. 12), distance 135 (e.g., as shown in FIG. 12), and a height 238 of a distal arch (e.g., a distance from a distal end of the frame body (e.g., a distal end of a lattice of the frame body or a distal end of a longitudinal strut of the frame body used to measure height 137) to a distal portion of a distal arch 116.

The material from which a portion of frame structure 12 (e.g., a strut 113, minimal valve support 124, leaflet 16, or fabric covering 112) is fabricated can impact the structural strength that the portion of frame structure 12 provides to frame structure 12 and/or valve prosthesis device 10. Valve prosthesis 10 or a portion thereof can comprise one or more materials that are sterilizable and/or biocompatible.

A portion of frame structure 12, such as strut 113 and/or minimal valve support 124 (e.g., hoop structure) that can be used to provide frame structure 12 with compressive strength and/or resiliency can be made of a metal or a metal alloy. Representative examples of metals and metal alloys that can be used to form all or part of a portion of frame structure 12 include nickel-titanium alloys (NiTi), cobalt-chrome alloys, and stainless steel. A portion of a frame structure (e.g., strut 113 or minimal valve support 124) can be made of a material comprising one or more of the following metals: titanium, aluminum, cobalt, chrome, molybdenum, vanadium, zirconium, zinc, nickel, niobium, tantalum, magnesium, and iron. Specific titanium alloys that can be used include Ti-3Al-2.5V, Ti-5Al-2.5Fe, Ti-6-Al-4V, Ti-6Al-4V ELI, Ti-6Al-7Nb, Ti-15Mo, Ti-13Nb-13Zr, Ti-12Mo-6Zr-2Fe, Ti-45Nb, Ti-35Nb-7Zr-5Ta, and Ti-55.8Ni. A portion of a frame structure 12 can comprise a nickel-titanium alloy having equal or nearly equal amounts of nickel and titanium. For example, a nickel-titanium alloy can be 50 mol %, from 49.5 mol % to 50.5 mol %, from 49 mol % to 51 mol %, from 48.5 mol % to 51.5 mol %, from 48 mol % to 52 mol %, 47.5 mol % to 52.5 mol %, or from 47 mol % to 53 mol % nickel.

In some cases, a portion of valve prosthesis 10 can comprise a ceramic. For example one or more portions of frame structure 12 can comprise one or more of alumina, zirconia, quartz, pyrolytic carbon (e.g., pyrolytic carbon coated graphite), or a calcium phosphate such as hydroxyapatite.

A portion of valve prosthesis 10 can comprise a polymer (e.g., a sterilizable polymer and/or biocompatible polymer). In some cases, a polymer can comprise one or more of polyethylene (e.g., polyethylene terephthalate (PET), polytetrafluoroethylene (PTFE)), a fluoropolymer, silicone, polystyrene, nylon, polyurethane, thermoplastic polyurethane (TPU), polysiloxane, polylactide (PLA), polyglycolide (PGA), poly(lactide-co-glycolide) (PLGA), polycaprolactone (PCL) such as poly(ε-caprolactone), poly(methyl methacrylate), hyaluronan, polydioxanone, polyanhidride, or trimethylene carbonate. In some cases, a polymer of a valve prosthesis 10 or portion thereof can be a co-polymer (e.g., a block co-polymer). In some cases, a polymer can be cross-linked (e.g., using ultraviolet light) to increase strength and/or resiliency of a polymer.

Materials comprising valve prosthesis 10 or a portion thereof (e.g., frame structure 12, fabric covering 112, or strut 113) can be formed into solid structures or meshes. For example, fabric covering 112 can comprise one or more materials (e.g., polymers such as polyester or nylon) formed into a fabric or mesh.

In some cases, valve prosthesis 10 or a portion thereof (e.g., valve leaflet 16) can comprise a cell-based tissue. The use of a cell-based tissue as a material for valve prosthesis 10 or a portion thereof can offer various advantages, such as decreased thrombogenicity, improved integration of an implanted valve prosthesis 10 with surrounding native tissue, improved material properties of the device or portion thereof, and, in some cases, decreased immune response. For example, a valve prosthesis 10 (or portion thereof) comprising a cell-based tissue can exhibit mechanical properties closer to those of a healthy valve under static and/or dynamic mechanical loading. A cell-based tissue derived from a subject's own tissue (e.g., stem-cell derived tissues) or from an allogenic source comprising all or a portion of valve prosthesis 10 can decrease the likelihood of immunogenic response after implantation, in some cases. In some cases, one or more cells of a cell-based tissue useful in a valve prosthesis 10 can be autologous, allogeneic, or xenogeneic relative to a subject in which the prosthetic valve device is deployed. Representative examples of sources of one or more cells of a cell-based tissue useful in a valve prosthesis 10 are a human, a pig, or a cow. One or more distal (or ventricular) surfaces of leaflet 16 can be fabricated from, coated with, or treated with a biocompatible material.

A system comprising valve prosthesis 10 can include a delivery device. The delivery device may comprise an outer sheath (e.g., an outer catheter), an inner shaft (e.g., a delivery tube) disposed within a lumen of the outer sheath, and/or an optional guidewire disposed within a lumen of the inner shaft. The guidewire may optionally comprise a nosecone to facilitate guidance of the guidewire to the native valve. A proximal end of the valve prosthesis 10 may be operably coupled to the inner shaft during delivery to the native valve as described herein. The outer sheath or inner sheath of the delivery device may be steerable.

The valve prosthesis 10 may be operably coupled to a delivery device. Additional description for the delivery device and other similar delivery devices usable in the embodiments described herein may be found in U.S. patent application Ser. No. 16/546,901, filed Aug. 21, 2019, entitled “Prosthetic Cardiac Valve Devices, Systems, and Methods; U.S. patent application Ser. No. 16/594,946, filed Oct. 7, 2019, entitled “Prosthetic Cardiac Valve Devices, Systems, and Methods”; International Patent Application No. PCT/US2019/057082, filed Oct. 18, 2019, entitled “Adjustable Medical Device”; U.S. patent application Ser. No. 16/723,537, filed Dec. 20, 2019, entitled “Prosthetic Cardiac Valve Devices, Systems, and Methods” and International Patent Application No. PCT/US2020/023671, filed Mar. 19, 2020, entitled “Prosthetic Cardiac Valve Devices, Systems, and Methods”; the entireties of which are incorporated by reference in their entireties, which are incorporated herein by reference for all purposes.

As would be understood by a person of skill in the art, various embodiments of valve segments, valve anchors, and frame anchors, can offer advantages for the treatment or replacement of a native valve.

Prosthetic valve devices described herein can be used to treat a subject (e.g., a patient) having a diseased heart valve. In some cases, treating a subject comprises repairing or replacing a valve of the subject, such as a heart valve (e.g., a mitral valve, a pulmonary valve, a tricuspid valve, or an aortic valve). A prosthetic valve device can be introduced into a subject transseptally or transapically (e.g., via a delivery device such as a catheter, which may comprise an outer sheath). In many cases, treating a subject (e.g., patient) can comprise positioning a prosthetic valve device described herein in a native valve of the subject. Structures and devices described herein (e.g., self-expandable, malecot-expandable, or balloon-expandable prosthetic valve devices and frame structures; anchors; barbs; hooks; coil grabbers) can be used to secure a prosthetic valve device in a target region or location, such as within a native valve of a subject.

The distal end of the delivery device may be configured to be advanced from a first side of a native valve to a second side of the native valve. For example, the distal end of the delivery device may be advanced from a left atrial side of a mitral valve to a left ventricular side of a mitral valve. In some instances, the distal end of the delivery device may be transseptally inserted into the left atrium of the heart prior to advancement into the left ventricle. Alternatively, or in combination, the distal end of the delivery device may be steerable such that it is positionable to point towards the first side of the native valve before being advanced to the second side of the native valve.

In some embodiments, fully deploying the anchor 15 may comprise actuating the anchor 15 from an elongated delivery configuration to a deployed configuration on the first side of the native valve and advancing the anchor 15 in the deployed configuration through the native valve to the second side of the native valve. Advancing the anchor 15 may comprise pushing the anchor through the native valve. Advancing the anchor 15 may further comprise rotating the anchor 15 through the native valve.

In some embodiments, fully deploying the anchor 15 may comprise positioning the anchor 15 such that it is located only on the second side of the native valve.

In some embodiments, the anchor 15 may be actuated from the delivery configuration to the deployed configuration on a first side of the native valve prior to being advanced to a second side of the native valve. For example, the anchor 15 may be deployed in a left atrium of a heart prior to being advanced to a left ventricle of the heart as described herein.

In some cases, the anchor 15 may be actuated from the delivery configuration to the deployed configuration on a second side of the native valve after being advanced to the second side from a first side of the native valve. For example, anchor 15 may be advanced from a left atrium of a heart prior to being deployed in a left ventricle of the heart.

In some cases, the anchor is deployed after the frame structure has been deployed in a target region of the subject. For example, an anchor can be advanced through an end (e.g., a distal end) of the frame structure when the frame structure is in an expanded configuration. Advancing anchor 15 can comprise pushing the anchor through the native valve and/or rotating the anchor. Anchor 15 can be configured (e.g., through a helical shape) to wrap at least partially around valve prosthesis 10 or a portion thereof. For example, anchor 15 can be configured to wrap at least partially around frame structure 12.

The free end 22 of the deployed anchor 15 may optionally be rotated around one or more structures on the second side of the native valve. The one or more structures may comprise one or more valve leaflets of the native valve. Alternatively, or in combination, the one or more structures may comprise one or more chordae of the left ventricle.

The free end 22 of the deployed anchor 15 may optionally rotated around one or more structures on the second side of the native valve such that the one or more structures (e.g., chordae, leaflets, or annulus) are pulled radially inwards towards the longitudinal axis of the anchor 15 and/or towards the longitudinal axis of the delivery device. The anchor 15 and/or free end 22 may be configured such that minimal torque is applied to the one or more structures. Alternatively, or in combination, the anchor 15 and/or free end 22 may be configured such that the one or more structures are not rotated, or are minimally rotated, during rotation of the anchor 15. Anchor 15 can comprise a proximal end opposite free end 22, which may be used to couple anchor 15 to prosthetic valve device 15 or a portion thereof.

The anchor 15 may then be released from the distal end of the delivery device. The anchor 15 may be released from the distal end of the delivery device on the second side of the native valve.

The frame structure 12 may be expanded within the native valve from an unexpanded configuration to an expanded configuration.

The frame structure 12 may be released from the distal end of a delivery device. In some embodiments, at least a portion the frame structure may be expanded within at least a portion of the deployed anchor to anchor the frame structure to the native valve. In some embodiments, expanding the frame structure and releasing the frame structure may occur simultaneously. Finally, the delivery device may be retracted from the native valve.

A delivery device may comprise an inner shaft as described herein. The delivery device may optionally comprise an outer shaft, a guidewire, and/or an inflatable balloon, in any combination thereof as desired by one of ordinary skill in the art.

A distal end of the delivery device may be inserted into the left atrium of the heart via a transseptal puncture as described herein. For example, the distal ends of inner shaft and/or outer sheath may be advanced into the left atrium of the heart. The inner shaft may optionally be advanced distally into the left atrium away from the distal end of the outer sheath. In some embodiments, advancing the inner shaft relative to the outer sheath may aid in deployment and/or placement of the valve prosthesis 10 as described herein. Alternatively, both the inner shaft and the outer sheath may be advanced distally into the left atrium through the transseptal puncture.

At least a portion of the valve prosthesis 10 may be deployed from an undeployed (for example, compressed or unexpanded) configuration to an expanded configuration within the left atrium. At least a portion of the anchor 15 may be deployed from a delivery and/or elongated configuration to a deployed configuration within the heart. For example anchor, may be actuated from an elongated configuration to a deployed configuration within the left atrium as described herein. In some embodiments, the anchor 15 may be deployed from the inner shaft by pushing the anchor 15 out of the inner shaft, releasing the anchor 15 from radial constraint by retracting the outer sheath, or the like as described herein. After the anchor 15 has been deployed from the delivery device 30, the frame structure 12 may be at least partially deployed from the delivery device so as to place the frame structure 12 within the anchor 15. The frame structure 12 may be deployed from the delivery device in either the unexpanded configuration or the expanded configuration, depending on the location of deployment, as will be understood by one of ordinary skill in the art.

The distal end of the delivery device (for example, the distal end of the inner shaft and/or the outer sheath) may be steered such that the distal end of the delivery device points toward the atrial side of the native valve. Such steering may occur prior to, during, or after deployment of at least a portion (for example deployment of an anchor 15) of the valve prosthesis 10. In some embodiments, the distal end of the outer sheath may be steerable. Alternatively, or in combination, the inner shaft may comprise a joint configured to change an angle of the distal portion of the inner shaft relative to a proximal portion of the inner shaft. The inner shaft may be steered by changing the angle of the distal portion of the inner shaft relative to the proximal portion of the inner shaft. The angle of the joint may be changed passively or actively. In various embodiments, the angle may be selectively controlled by a proximal handle. For example, pull wires or other mechanisms may connect to the joint to controls on the handle.

Valve prosthesis 10 may be advanced through the native valve by the delivery device from the left atrium to the left ventricle. Advancement of the valve prosthesis 10 and optionally delivery device through the mitral valve may be facilitated by the natural opening and closing of the valve during the cardiac cycle. The distal end of the delivery device and/or valve prosthesis 10 may be configured to be advanced from a first side of a native valve to a second side of the native valve. For example, the distal end of the delivery device and/or valve prosthesis 10 may be advanced from a left atrial side of a mitral valve to a left ventricular side of a mitral valve. Advancing the anchor 15 may comprise pushing the anchor 15 through the native valve. Alternatively, or in combination, advancing the anchor 15 may comprise rotating the anchor 15 through the native valve. In some instance, the combination of rotational motion and pushing may facilitate advancement of the device from the first side of the native valve to the second side of the native valve. Rotation of the valve prosthesis device 10, for example rotation of the anchor 15 and/or frame structure 12, may be facilitated by the inner shaft. For example, the inner shaft may transmit rotational motion to the valve prosthesis 10 in order to rotate the valve prosthesis 10 during advancement through the native valve.

Rotation of the anchor 15 during advancement may assist with the stretching process by aiding in unwinding the anchor 15. Additionally, the rotational motion may reduce the risk of the free end 22 of the anchor 15 undesirably engaging other anatomy during insertion through the native valve leaflets. The anchor 15 may be sufficiently elastic so as to enable relatively easy insertion through the native valve and/or reduce the risk of injury to the native leaflets. After the anchor 15 has stretched through the native valve it may return to the deployed configuration.

In some embodiments, the anchor 15 may be advanced to the ventricle before being deployed from the delivery (e.g., elongated) configuration to the deployed configuration.

One or more structures on the ventricular side of the native valve may comprise one or more valve leaflets and/or one or more chordae tendineae. After the anchor 15 has been at least partially deployed within the left ventricle adjacent one or more chordae tendineae, the valve prosthesis 10 may be rotated to capture and anchor the native chordae and/or native leaflets. The free end 22 of the anchor 15 may extend radially outward from the rest of the anchor 15 to facilitate capture of the native structures. The free end 22 of the coil 15 may be rotated around one or more of the chordae tendineae. Additional rotation of the valve coil 15 may gradually capture additional chordae tendineae.

Rotation of the valve prosthesis 10, for example, rotation of the anchor 15 and/or frame structure 12, may be facilitated by the delivery device. For example, the inner shaft may be rotated and rotational motion may be transmitted from the inner shaft to the valve prosthesis 10 in order to rotate the valve prosthesis 10 around one or more of the structures on the ventricle side of the mitral valve as described herein.

A portion of valve prosthesis 10 may be wrapped around the captured chordae tendineae. The valve prosthesis 10 may be rotated around the chordae tendineae such that the chordae tendineae are pulled inwardly into bunches. The native valve leaflets may also be in communication with the valve prosthesis device 10. The valve prosthesis 10 may be rotated to capture enough chordae tendineae and/or valve leaflets to rigidly anchor the anchor 15 adjacent the native valve annulus. The valve prosthesis device 10 may be anchored by wrapping around only a portion of the chordae. Although it may be possible to capture all or substantially all the chordae 40, this may not be necessary to provide sufficient anchoring of the valve prosthesis device 10. The prosthesis may be further anchored by expansion of the frame structure 12 within the native valve and against the anchor 15.

Valve prosthesis 10 can be anchored to a structure of the native tissue environment (e.g., a native valve or portion thereof, one or more chordae tendinae, or wall of a heart chamber) by engaging one or more hooks, barbs, and/or scallop-shaped anchors coupled to valve prosthesis 10 with the structure of the native tissue environment (e.g., a portion of a native valve). For example, valve prosthesis 10 may be secured at a target region of a subject by engaging the frame structure 12 with the anchor 15 and/or via one or more barbs of the valve prosthesis device interacting a structure of the native tissue environment.

Once the anchor 15 has been anchored adjacent to the native valve, the frame structure 12 and prosthetic valve segment 14 may be expanded at least partially within the anchor 15 as described herein. The frame structure 12 and the valve segment 14 may be deployed (e.g., expanded) simultaneously. Alternatively, or in combination, the frame structure 12 and the valve segment 14 may be deployed sequentially, for example by first expanding the frame structure 12 and then receiving the prosthetic valve segment 14 therein.

Frame structure 12 may be expanded within the native valve from an unexpanded configuration to an expanded configuration. In some embodiments, at least a portion the frame structure 12 may be expanded within at least a portion of the deployed anchor 15 to anchor the frame structure 12 to the native valve. In some embodiments, the frame structure 12 may comprise an expandable stent. In some embodiments, the frame structure 12 of valve prosthesis 10 may be self-expandable (e.g., self-expanding). In some embodiments, the frame structure 12 of valve prosthesis 10 may be balloon-expandable. The delivery device may comprise a balloon which may be disposed within the valve prosthesis 10 in order to expand the valve prosthesis device 10. The balloon may be positioned within at least a portion of the valve prosthesis device 10, for example within at least a portion of frame structure 12 in an uninflated configuration prior to being inflated. The inflatable balloon may, for example, be disposed within the inner shaft or outer sheath of the delivery device while the anchor 15 is being positioned adjacent the native valve and then advanced therefrom (or the inner shaft or outer sheath is retracted therefrom) to be positioned within the frame structure 12. Alternatively, the inflatable balloon may be disposed within the frame structure 12 during placement of the valve prosthesis 10. Frame structure 12 may be partially expanded towards the anchor 15 in order to capture the chordae tendineae therebetween. As the frame structure 12 continues to be expanded to a fully expanded state the chordae tendineae may be sandwiched between the anchor 15 and the frame structure 12. The frame structure 12 and anchor 15 may thus be anchored to the chordae tendineae.

The valve prosthesis 10 may then be released from the delivery device. In some embodiments, releasing the valve prosthesis 10 may comprise releasing the anchor 15 and/or the frame structure 12. Releasing the valve prosthesis 10 from the delivery device may comprise expanding the valve prosthesis 10 from the unexpanded configuration to the expanded configuration. For example, expanding the frame structure 12 and releasing the frame structure 12 may occur simultaneously as described herein. Alternatively, the frame structure 12 may be released prior to or after being expanded.

A method may further comprise deflation of the balloon, retraction of the balloon into inner shaft, and/or removal of the delivery device from the heart. After the frame structure 12 has been expanded and anchored to the native valve as described herein, the inflatable balloon may be deflated. The balloon may optionally be retracted back into the delivery device, for example into inner shaft. The delivery device may then be removed from the heart.

Although the steps above show a method of deploying a valve prosthesis 10 within a native valve in accordance with embodiments, a person of ordinary skill in the art will recognize many variations based on the teaching described herein. The steps may be completed in a different order. Steps may be added or deleted. Some of the steps may comprise sub-steps. Many of the steps may be repeated as often as necessary to assemble at least a part of an article.

For example, in some embodiments deploying the valve prosthesis 10 may occur in multiple steps such that a portion of the valve prosthesis 10 (e.g., anchor 15) may be deployed before another portion the valve prosthesis 10 (e.g., frame structure 12). Alternatively, or in combination, in some embodiments, deploying the anchor 15 may occur in multiple steps such that a portion of the anchor 15 may be deployed before being advanced through the native valve and another portion of the anchor 15 may be deployed after being advanced through the native valve. Alternatively, or in combination, the delivery device may be advanced from the left atrium to the left ventricle with the valve prosthesis 10 undeployed. In many embodiments, the frame structure may 12 be self-expanding and the balloon may not be necessary for expansion of the frame structure 12. Alternatively, or in combination, the anchor 15 may be released after the frame structure 12 has been expanded within it.

It should be understood that any feature described herein with respect to one embodiment can be substituted for or combined with any feature described with respect to another embodiment. For example, the features described with respect to of any of valve prostheses 10, 10A, 10B, 10C, 10D, 10E, 10F, 10G, 10H, 10I, 10J can be exchanged for or combined with respect to any of the other valve prostheses 10, 10A, 10B, 10C, 10D, 10E, 10F, 10G, 10H, 10I, 10J.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A device for treating a diseased native valve in a patient, the device comprising:
a frame structure having an unexpanded configuration and an expanded configuration, the frame structure in the expanded configuration comprising a first end portion, a central annular portion, and a second end portion; and
a valve segment coupled to the central annular portion and at least partially longitudinally aligned with the first end portion when the frame structure is in the expanded configuration, the valve segment comprising a biocompatible one-way valve;
wherein an inflow edge of the valve segment is unsupported by the first end portion of the frame structure and spaced radially inwards from the first end portion when the frame structure is in the expanded configuration.

2. The device of claim 1, wherein the frame structure has a longitudinal length of less than 35 mm in the expanded configuration.

3. The device of claim 1, wherein the valve segment comprises a plurality of leaflets.

4. The device of claim 3, wherein the valve segment further comprises a seal positioned radially between the frame structure and the plurality of leaflets.

5. The device of claim 4, wherein the seal is attached to the central annular portion of the frame structure.

6. The device of claim 3, wherein the plurality of leaflets are attached to the frame structure only at commissures of the leaflets.

7. The device of claim 3, wherein the plurality of leaflets are supported at a nadir of each leaflet by a nadir support extending from the central annular portion.

8. The device of claim 1, wherein at least portion of the inflow edge extends beyond the frame structure while an entire outflow edge of the valve segment is positioned within the frame structure.

9. The device of claim 1, wherein the first end portion flares radially outwards from the central annular portion.

10. The device of claim 1, wherein the second end portion flares radially outwards from the central annular portion.

11. The device of claim 1, wherein the first and second end portions are configured to engage an exterior anchor therebetween when the frame structure is in the expanded configuration.

12. The device of claim 1, wherein the central portion is configured to apply outward radial pressure to engage with an exterior anchor.

13. The device of claim 1, wherein the frame structure is configured to self-expand from the unexpanded configuration to the expanded configuration.

14. The device of claim 1, wherein the frame structure is configured to couple to the native valve such that the first end portion is oriented toward an atrial side of the native valve and the second end portion is oriented toward a ventricular side of the native valve.

15. The device of claim 1, wherein the diameter of the frame structure in the expanded configuration is between 25 mm and 35 mm.

16. A device for treating a diseased native valve in a patient, the device comprising:
a frame structure having an unexpanded configuration and an expanded configuration; and
a valve segment coupled to the frame structure, the valve segment comprising a biocompatible one-way valve, the valve segment further having an inflow edge and an outflow edge, wherein a majority of the inflow edge of the valve segment is unsupported by the frame structure;
wherein an inflow end of the frame structure comprises a plurality of flared flanges extending radially therefrom, the inflow edge of the valve segment positioned radially inwards of the plurality of flared flanges when the frame structure is in the expanded configuration.

17. The device of claim 16, wherein the frame structure has a longitudinal length of less than 35 mm in the expanded configuration.

18. The device of claim 16, wherein the valve segment comprises a plurality of leaflets.

19. The device of claim 18, wherein the valve segment further comprises a seal positioned radially between the frame structure and the plurality of leaflets.

20. The device of claim 19, wherein the seal is attached to a central portion of the frame structure.

21. The device of claim 18, wherein the plurality of leaflets are attached to the frame structure only at commissures of the leaflets.

22. The device of claim 18, wherein the plurality of leaflets are supported at a nadir of each leaflet by a nadir support extending from the frame structure.

23. The device of claim 16, wherein at least portion of the inflow edge extends beyond the frame structure while an entire outflow edge of the valve segment is positioned within the frame structure.

24. The device of claim 16, wherein an outflow end of the frame structure comprises a plurality of flared flanges extending radially therefrom.

25. The device of claim 24, wherein the inflow end and the outflow end are configured to engage an exterior anchor therebetween when the frame structure is in the expanded configuration.

26. The device of claim 16, wherein the frame structure comprise a central annular portion configured to apply outward radial pressure to engage with an exterior anchor.

27. The device of claim 16, wherein the frame structure is configured to self-expand from the unexpanded configuration to the expanded configuration.

28. The device of claim 16, wherein the frame structure is configured to couple to the native valve such that the inflow edge is oriented toward an atrial side of the native valve and the outflow edge is oriented toward a ventricular side of the native valve.

29. The device of claim 16, wherein the diameter of the frame structure in the expanded configuration is between 25 mm and 35 mm.

30-69. (canceled)
